(19) United States
(12) Patent Application Publication
(10) Pub. No.: US 2019/0382751 A1
(43) Pub. Date: Dec. 19, 2019

(54) MODIFIED CRISPR RNA AND USES THEREOF

(71) Applicants: Ionis Pharmaceuticals, Inc., Carlsbad, CA (US); Ludwig Institute for Cancer Research, New York, NY (US)

(72) Inventors: Meghdad Radhar, Cardiff by the Sea, CA (US); Thazha P. Prakash, Carlsbad, CA (US); Eric E. Swayze, Encinitas, CA (US); C. Frank Bennett, Carlsbad, CA (US); Moira A. McMahon, Cardiff by the Sea, CA (US)

(73) Assignees: Ionis Pharmaceuticals, Inc., Carlsbad, CA (US); Ludwig Institute for Cancer Research, New York, NY (US)

(21) Appl. No.: 16/470,464

(22) PCT Filed: Dec. 28, 2017
(86) PCT No.: PCT/US17/68642

§ 371 Date: Jun. 17, 2019

Related U.S. Application Data
(60) Provisional application No. 62/439,828, filed on Dec. 28, 2016.
(60) Provisional application No. 62/491,818, filed on Apr. 28, 2017.
(60) Provisional application No. 62/572,361, filed on Oct. 13, 2017.

(51) Int. Cl. C12N 15/10; C12N 15/11

(52) U.S. Cl. CPC C12N 15/102; C12N 15/111; C12N 2310/20; C12N 2310/321; C12N 2310/315; C12N 2310/3533; C12N 2310/346; C12N 2320/51; C12N 2310/3521; C12N 2310/322; C12N 2310/3231

(57) ABSTRACT

The present disclosure provides compounds comprising modified oligonucleotides for use in CRISPR. In certain embodiments, such modified oligonucleotides provide improved properties of crRNA.

### SEQUENCE LISTING

The present application is being filed along with a Sequence Listing in electronic format. The Sequence Listing is provided as a file entitled CORE0141WOSEQ_ST25.txt, created on Dec. 15, 2017, which is 948 Kb in size. The information in the electronic format of the sequence listing is incorporated herein by reference in its entirety.

### BACKGROUND

Use of Cluster Regulatory Interspaced Short Palindromic Repeats (CRISPR) to edit or disable genes has been described. See for example Jinek et al., Science 337: 816-821 (2012); Mali et al. Science 339: 823-826 (2013).

### SUMMARY

Various CRISPR systems have been described. See for example: WO2013/176772; WO2015/006747; Qi et al., Cell 152: 1 173-1 (2013); Gilbert et al., Cell 154: 1-10 (2013) Jinek et al., Science 337: 816-821 (2012); Mali et al. Science 339: 823-826 (2013); Doudna et al., Science 346: 6213 (2014). See also for example: Zetsche et al., Cell 163: 1-13 (2015). The present invention provides modified oligonucleotides for use as crRNA in CRISPR systems. In certain embodiments, such modified crRNA have improved stability relative to unmodified crRNA. In certain embodiments, modified crRNA is stabilized at the 5′ end and/or the 3′. In certain embodiments, such stabilized crRNA is resistant to exonuclease and/or endonucleoase digestion. In certain embodiments, modified crRNA have improved affinity for target DNA or RNA relative to unmodified crRNA. In certain embodiments, modified crRNA have improved selectivity for target DNA or RNA relative to unmodified crRNA. In certain embodiments, modified crRNA have improved cellular uptake relative to unmodified crRNA. In certain embodiments, modified crRNA increase gene editing activity of a CRISPR system relative to unmodified crRNA.

In certain embodiments, the crRNA modifications increase affinity for the target DNA or RNA allowing the modified crRNA to be shortened while retaining sufficient affinity to hybridize to target DNA or RNA and/or associate with other CRISPR system components. Thus, in certain embodiments, modified crRNA is shorter than unmodified crRNA. In certain embodiments, modified crRNA is 35-45 linked nucleosides in length. In certain embodiments, modified crRNA is 35-43 linked nucleosides in length. In certain embodiments, modified crRNA is 35-42 linked nucleosides in length. In certain embodiments, modified crRNA is 36-43 linked nucleosides in length. In certain embodiments, modified crRNA is 36-42 linked nucleosides in length. In certain embodiments, modified crRNA is 36-40 linked nucleosides in length. In certain embodiments, the target recognition portion of modified crRNA is 15-23 linked nucleosides in length. In certain embodiments, the target recognition portion of modified crRNA is 15-22 linked nucleosides in length. In certain embodiments, the target recognition portion of modified crRNA is 16-22 linked nucleosides in length. In certain embodiments, the target recognition portion of modified crRNA is 17-22 linked nucleosides in length. In certain embodiments, the target recognition portion of modified crRNA is 18-22 linked nucleosides in length. In certain embodiments, the target recognition portion of modified crRNA is 16-20 linked nucleosides in length. In certain embodiments, the target recognition portion of modified crRNA is 18-20 linked nucleosides in length. In certain embodiments, the CRISPR recognition portion of modified crRNA is 17-20 linked nucleosides in length. In certain embodiments, the CRISPR recognition portion of modified crRNA is 18-20 linked nucleosides in length. In certain embodiments, such shorter crRNA have improved uptake properties and/or are easier to synthesize than longer crRNA. In certain embodiments, modified crRNA are taken into cells without transfection reagents or electroporation. In certain such embodiments, the cells are in an animal. In certain embodiments, the animal expresses a CRISPR nuclease. In certain embodiments, the animal is previously or concomitantly treated with a means of expressing a CRISPR nuclease. In certain such embodiments, such treatment comprises administration of a vector for delivering a CRISPR nuclease. In certain such embodiments, such vector is a viral vector, for example adeno-associated virus (AAV). In certain such embodiments, the viral vector expresses a bacterial derived CRISPR nuclease that fits into an AAV vector. In certain embodiments, the CRISPR nuclease is a Cpf1 nuclease.

In certain embodiments, the CRISPR system is inhibited after the target gene is edited. In certain such embodiments, the modified crRNA inside a cell is degraded after the target gene has been edited. In certain such embodiments, the CRISPR nuclease continues to be expressed in the cell but is no longer active because it requires crRNA in order to exhibit nuclease activity. In certain such embodiments, off-target effects of the CRISPR system, such as undesired cleavage of an off-target gene, are decreased relative to a CRISPR system in which all of the components necessary for nuclease activity continue to be expressed indefinitely, e.g. by a viral vector. In certain such embodiments, degradation of the modified crRNA is facilitated by hybridization to an oligonucleotide complementary to the crRNA. In certain embodiments, degradation of the modified crRNA is facilitated by nucleases present in the cell.

In certain embodiments, the CRISPR system is inhibited after the target gene is edited via degradation of a tracrRNA inside the cell. In certain such embodiments, degradation of the tracrRNA is facilitated by hybridization to an oligonucleotide complementary to the tracrRNA. In certain embodiments, degradation of the tracrRNA is facilitated by nucleases present in the cell.

In certain embodiments, the CRISPR system is inhibited after the target gene is edited via inhibition of the expression of the CRISPR nuclease. In certain such embodiments, the nuclease gene is edited by a modified crRNA. In certain embodiments, the nuclease transcript is degraded following hybridization of the nuclease transcript to an oligonucleotide complementary to the nuclease transcript.

### BRIEF DESCRIPTION OF FIGURES

FIG. 1 shows a gel that illustrates the extent of gene editing of DNMT1 by modified crRNAs described in Example 12.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### Definitions

Unless otherwise indicated, the following terms have the following meanings:

As used herein, “2′-deoxynucleoside” means a nucleoside comprising 2′-H(H) furanosyl sugar moiety, as found in naturally occurring deoxyribonucleic acids (DNA). In a crRNA, a 2′-deoxynucleoside is a modified nucleoside.

As used herein, “2′-substituted nucleoside” or “2-modified nucleoside” means a nucleoside comprising a 2′-substituted or 2′-modified sugar moiety. As used herein, “2′-substituted” or “2-modified” in reference to a sugar moiety in a crRNA means a furanosyl sugar moiety comprising a 2′-substituent group in place of the 2′-OH of an unmodified sugar moiety.

As used herein, “3′-stabilized” in reference to a modified oligonucleotide means a modified oligonucleotide that comprises at least one stabilizing modification or is connected to a stabilizing conjugate group, wherein the at least one modification and/or the conjugate group increases the stability of the 3′-terminus of the modified oligonucleotide in cells or in an animal relative to a corresponding oligonucleotide that does not comprise the at least one stabilizing modification or is not connected to the stabilizing conjugate group. In certain embodiments, modified crRNAs are 3′-stabilized. In certain such embodiments, the 3′-terminal nucleoside of the modified crRNA comprises the stabilizing modification. In certain embodiments, the 3′-terminal internucleoside linkage of the crRNA comprises the stabilizing modification.

As used herein, “5′-stabilized” in reference to a modified oligonucleotide means a modified oligonucleotide that comprises at least one stabilizing modification or is connected to a stabilizing conjugate group, wherein the at least one modification and/or the conjugate group increases the stability of the 5′-terminus of the modified oligonucleotide in cells or in an animal relative to a corresponding oligonucleotide that does not comprise the at least one stabilizing modification or is not connected to the stabilizing conjugate group. In certain embodiments, modified crRNAs are 5′-stabilized. In certain such embodiments, the 5′-terminal nucleoside of the modified crRNA comprises the stabilizing modification. In certain such embodiments, the 5′-terminal nucleoside of the modified crRNA is a linker nucleoside.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “cell-targeting moiety” means a conjugate group or portion of a conjugate group that is capable of binding to a particular cell type or particular cell types.

As used herein, “complementary” in reference to an oligonucleotide means the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to adenine (A) and thymine (T), adenine (A) and uracil (U), cytosine (C) and guanine (G), and 5-methyl cytosine (^{m}C) and guanine (G) unless otherwise specified. Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside. In such embodiments, mismatches are not tolerated.

As used herein, “conjugate group” means a group of atoms that is directly or indirectly attached to a parent compound, e.g., an oligonucleotide.

As used herein, “conjugate linker” means a group of atoms that connects a conjugate group to a parent compound, e.g., an oligonucleotide.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other

As used herein, “crRNA” or “CRISPR RNA” means an oligonucleotide that comprises a target recognition portion and a CRISPR recognition portion. As used herein, a “target recognition portion” is a portion of an oligonucleotide with a nucleobase sequence that is complementary to a DNA or RNA target. As used herein, “CRISPR recognition portion” is a portion of an oligonucleotide that can bind to, associate with, or contribute to the binding or association with a CRISPR nuclease or a molecule that binds to or associates with a CRISPR nuclease. The CRISPR recognition portion of a crRNA is not complementary to the DNA or RNA target of the target recognition portion of the crRNA. Thus, although the target recognition portion of a crRNA may associate with a CRISPR nuclease, the target recognition and CRISPR recognition portions of a crRNA do not overlap. A CRISPR nuclease is a protein that directly or indirectly associates with a crRNA and cleaves the target DNA or RNA. In certain embodiments, the CRISPR nuclease is a Cpf1 nuclease. In certain such embodiments, the CRISPR recognition portion of a crRNA binds to or associates with a Cpf1 nuclease. In certain embodiments, the CRISPR recognition portion of a crRNA binds to or associates with a tracrRNA. In certain embodiments, crRNAs comprise a self-complementary region. In certain such embodiments, the CRISPR recognition portion partially or completely overlaps with the self-complementary region. In certain embodiments, crRNAs comprise one or more linker nucleosides.

As used herein, “linker nucleosides” in the context of a crRNA means one or more nucleosides that are linked to the target recognition portion and/or the CRISPR recognition portion of a crRNA. Linker nucleosides are not part of either the target recognition portion or the CRISPR recognition portion of the crRNA. In certain embodiments, such linker nucleosides are located at the 5′-terminus of a crRNA, the 3′-terminus of the crRNA, and/or in between the target recognition and CRISPR recognition portions of a crRNA.

As used herein, “fully modified” in reference to an oligonucleotide means a modified oligonucleotide in which each sugar moiety is modified. “Uniformly modified” in reference to an oligonucleotide means a fully modified oligonucleotide in which each at least one modification of each sugar moiety is the same. For example, the nucleosides of a uniformly modified oligonucleotide can each have a 2′-MOE modification but different nucleobase modifications, and the internucleoside linkages may be different.

As used herein, “gene editing” means any process mediated by a complex comprising a CRISPR nuclease and a modified or unmodified crRNA, including but not limited to gene knock-down, gene knock-out, gene disruption, deletion, insertion, and gene activation.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “increases”, when used in reference to an effect mediated by a modified oligonucleotide, means that the effect is greater in the presence of the oligonucleotide containing a certain modification than the effect is in the presence of a corresponding oligonucleotide that does not contain the certain modification.

As used herein, the terms “internucleoside linkage” means a group that forms a covalent linkage between adjacent nucleosides in an oligonucleotide. As used herein “modified internucleoside linkage” means any internucleoside linkage other than a naturally occurring, phosphate internucleoside linkage. Naturally occurring, non-phosphate linkages are referred to herein as modified internucleoside linkages. “Phosphorothioate linkage” means a linkage between nucleosides wherein the phosphodiester bond of a phosphate linkage is modified by replacing one of the non-bridging oxygen atoms with a sulfur atom. A phosphorothioate linkage is a modified internucleoside linkage.

As used herein, “linearly modified sugar” or “linearly modified sugar moiety” means a modified sugar moiety that comprises an acyclic or non-bridging modification. Such linear modifications are distinct from bicyclic sugar modifications.

As used herein, “linked nucleosides” are nucleosides that are connected in a continuous sequence (i.e. no additional nucleosides are present between those that are linked) and are linked by internucleoside linkages.

As used herein, “mismatch” or means a nucleobase of a first oligonucleotide that is not capable of pairing with the corresponding nucleobase of a second oligonucleotide or target nucleic acid when the first and second oligomeric compound are aligned.

As used herein, “MOE” means methoxyethyl. “2′-MOE” means a —OCH₂CH₂OCH₃ group at the 2′ position of a furanosyl ring.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means a heterocyclic moiety capable of pairing with a second, different nucleobase. As used herein, “nucleobase sequence” means the order of contiguous nucleobases independent of any sugar or internucleoside linkage modification. As used herein, “modified nucleobase” means a nucleobase other than adenine (A), thymine (T), cytosine (C), uracil (U), and guanine (G), herein defined as the five, unmodified nucleobases. A universal base is a nucleobase that can pair with any one of the five unmodified nucleobases.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject.

As used herein “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein “pharmaceutical composition” means a mixture of substances suitable for administering to a subject. For example, a pharmaceutical composition may comprise an crRNA compound and a sterile aqueous solution. In certain embodiments, a pharmaceutical composition shows activity in free uptake assay in certain cell lines.

As used herein, “phosphorus moiety” means a group of atoms comprising a phosphorus atom. In certain embodiments, a phosphorus moiety comprises a mono-, di-, or tri-phosphate, or phosphorothioate.

As used herein “prodrug” means a therapeutic agent in an inactive form that is converted to an active form within the body or cells thereof by the action of endogenous enzymes or other chemicals and/or physiologic conditions.

As used herein, “self-complementary” in reference to an oligonucleotide means an oligonucleotide that is at least partially complementary to itself. In certain embodiments, a self-complementary oligonucleotide forms a hairpin when a portion of the self-complementary oligonucleotide hybridizes to itself.

As used herein, “sugar moiety” means a group of atoms that can link a nucleobase to another group, such as an internucleoside linkage, conjugate group, or terminal group. In certain embodiments, a sugar moiety is attached to a nucleobase to form a nucleoside. As used herein, “unmodified sugar moiety” in the context of crRNA means a 2′-OH(H) furanosyl moiety, as found in RNA. Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a sugar surrogate or a furanosyl moiety comprising any substitution relative to an unmodified sugar moiety. In certain embodiments, a modified sugar moiety is a 2′-substituted sugar moiety. Such modified sugar moieties include bicyclic sugars and linearly modified sugars.

As used herein, “sugar surrogate” means a modified sugar moiety having other than a furanosyl moiety that can link a nucleobase to another group, such as an internucleoside linkage, conjugate group, or terminal group. Modified nucleosides comprising sugar surrogates can be incorporated into one or more positions within an oligonucleotide. In certain embodiments, such oligonucleotides are capable of hybridizing to complementary oligomeric compounds or nucleic acids.

As used herein, “target nucleic acid,” “target RNA”, “target DNA,” “target gene” and “nucleic acid target” mean a nucleic acid to which the target recognition portion of a crRNA is complementary. In certain such embodiments, a crRNA is designed to affect the target nucleic acid. An “off-target gene” is a gene that a crRNA is not designed to affect. In certain embodiments, the editing of an off-target gene is deleterious.

As used herein, “terminal group” means a chemical group or group of atoms that is covalently linked to a terminus of an oligonucleotide.

### CRISPR Systems and Certain Oligonucleotides for Use in CRISPR Systems

1. Certain CRISPR RNA (crRNA)

In certain embodiments, the present invention provides modified oligonucleotides for use in CRISPR. Typically, CRISPR employs CRISPR RNA (crRNA), which hybridizes to target DNA or RNA and directly or indirectly recruits a nuclease that cleaves the target DNA or RNA. Thus, the crRNA in such systems has two functions: (1) recognition and hybridization to the target DNA or RNA and (2) recognition by a CRISPR nuclease or a molecule that recruits a CRISPR nuclease. Typically, in such systems, the crRNA comprises two portions which correspond to these two functions: a target recognition portion and a CRISPR recognition portion. The present invention provides modified oligonulcleotides that may be used as crRNA. Such modified oligonucleotides may have modifications in the target recognition portion and/or CRISPR recognition portion.

In certain embodiments, the CRISPR recognition portion of the crRNA comprises a portion of the direct repeat sequence from a bacterial organism that has a Cpf1 nuclease or a Cpf1 ortholog. In certain such embodiments, the CRISPR recognition portion of the crRNA comprises a sequence selected from the table below. In certain embodiments, the CRISPR recognition portion of the crRNA comprises 16, 17, 18, 19, or 20 nucleobases of a sequence selected from the table below. In certain embodiments, the CRISPR recognition portion of the crRNA consists of 16, 17, 18, 19, or 20 nucleobases of a sequence selected from the table below.

TABLE A
Direct repeat sequences used in CRISPR recognition portions of crRNA

| Organism | Sequence | SEQ ID NO. |
|---|---|---|
| Francisella novicida | UAAUUUCUACUGUUGUAGAU | 3 |
| Lachnospimceae bacterium MC2017 | AGAAAUGCAUGGUUCUCAUGC | 4 |
| Butyrivibrio proteoclasticus | AAAAUUACCUAGUAAUUAGGU | 5 |
| Peregrinibacteria bacterium | GGAUUUCUACUUUUGUAGAU | 6 |
| Parcubacteria bacterium | AAAUUUCUACUUUUGUAGAU | 7 |
| Smithella | GUUUCAAUCCACGCGCCCACGCGG | 8 |
| | GGCGCGAC | |
| Acidaminococcus | UAAUUUCUACUCUUGUAGAU | 9 |
| Lachnospimceae bacterium MA2020 | GAAUUUCUACUAUUGUAGAU | 10 |
| Candidatus Methanoplasma termitum | GAAUCUCUACUCUUUGUAGAU | 11 |
| Eubacterium eligens | UAAUUUCUACUUUGUAGAU | 12 |
| Moraxella bovoculi | AAAUUUCUACUGUUUGUAGAU | 13 |
| Leptospim inadai | GAAUUUCUACUUUUGUAGAU | 14 |
| Lachnospiraceae bacterium ND2006 | UAAUUUCUACUAAGUGUAGAU | 15 |
| Polphyromonas crevioricanis | UAAUUUCUACUAUUGUAGAU | 16 |
| Prevotella disiens | UAAUUUCUACUUCGGUAGAU | 17 |
| Polphyromonas macacae | UAAUUUCUACUAUUGUAGAU | 16 |

In certain embodiments, the target recognition portion of the crRNA comprises a nucleobase sequence that is complementary to a target DNA. In certain such embodiments, the entire nucleobase sequence of the target recognition portion is complementary to a target DNA. In certain embodiments, the nucleobase sequence of the target recognition portion is at least 80%, at least 85%, at least 90%, at least 95%, or 100% complementary to a target DNA. In certain embodiments, the target DNA is a DNMT1 gene. In certain embodiments, the nucleobase sequence of the target DNMT1 gene is GENBANK Accession Number NT_011295.10 truncated from nucleobases 1506424 to 1569013, herein referred to as SEQ ID NO: 66. In certain embodiments, the target DNA is a GRIN2B gene. In certain embodiments, the nucleobase sequence of the target GRIN2B gene is GENBANK Accession Number NC_000012.12 truncated from nucleobases 13534001 to 13985000, herein referred to as SEQ ID NO: 67. In certain embodiments, the target DNA is a LDLR gene. In certain embodiments, the nucleobase sequence of the target LDLR gene is GENBANK Accession Number NC_000019.10 truncated from nucleobases 11086001 to 11137000, herein referred to as SEQ ID NO: 68. In certain embodiments, the target DNA is a complement component 5 (C5) gene. In certain embodiments, the nucleobase sequence of the target C5 gene is GENBANK Accession Number NC_000009.12 truncated from nucleobases 120949001 to 12078000, herein referred to as SEQ ID NO: 69. In certain embodiments, the target DNA is an empty spiracles homolog1 (EMX1) gene. In certain embodiments, the nucleobase sequence of the target EMX1 gene is GENBANK Accession Number NC_000002.12 truncated from nucleobases 72908001 to 72940000, herein referred to as SEQ ID NO: 70.

In certain embodiments, modified crRNA comprises a target recognition portion, a CRISPR recognition portion, and linker nucleosides. The linker nucleosides are not part of the target recognition or CRISPR recognition portions of the crRNA. In certain embodiments, linker nucleosides are modified in order to provide nuclease stability. In certain such embodiments, the linker nucleosides provide exonuclease stability. In certain embodiments, modified crRNA contain no linker nucleosides. In certain embodiments, modified crRNA comprise 2 linker nucleosides.

In certain embodiments, modified crRNA comprise a CRISPR recognition portion, a target recognition portion, and optional, one or more linker nucleosides. The CRISPR recognition and target recognition portions and the optional linker nucleosides may be in any orientation relative to each other, as shown below, wherein “CR” is the CRISPR recognition portion, “Ta” is the target recognition portion, and “Ln” is the linker nucleoside(s). In certain embodiments, modified crRNA are represented by one of the following formulas:

5′-CR-Ta-3′
5′-Ln-CR-Ta-3′
5′-CR-Ta-Ln-3′
5′-Ln-CR-Ta-Ln-3′
5′-CR-Ln-Ta-3′
5′-Ln-CR-Ln-Ta-3′
5′-Ln-CR-Ln-Ta-Ln-3′
5′-Ta-CR-3′
5′-Ta-CR-Ln-3′
5′-Ln-Ta-CR-3′
5′-Ln-Ta-CR-Ln-3′
5′-Ta-Ln-CR-Ln-3′
5′-Ta-Ln-CR-3′
5′-Ln-Ta-Ln-CR-Ln-3′

In certain embodiments, a compound comprising a modified crRNA comprises a conjugate group. In certain such embodiments, the conjugate group is connected to the 5′-terminus of the modified crRNA. In certain embodiments, the conjugate group is connected to the 3′-terminus of the modified crRNA. In certain embodiments, the conjugate group is connected to an internal nucleoside or internucleoside linkage of the modified crRNA.

In certain embodiments, the modified crRNA is 5′-stabilized and/or 3′-stabilized. In certain such embodiments, the 5′- or 3′-terminal nucleoside of the 5′- or 3′-stabilized crRNA comprises a stabilizing modification, respectively. In certain embodiments, the nucleoside comprising the stabilizing modification is the terminal nucleoside of the CRISPR recognition portion. In certain embodiments, the nucleoside comprising the stabilizing modification is the terminal nucleoside of the target recognition portion. In certain embodiments, the nucleoside comprising the stabilizing modification is a linker nucleoside. In certain embodiments, the 5′- or 3′-stabilized crRNA is connected to a stabilizing conjugate group at the 5′- or 3′-terminus, respectively. In certain such embodiments, the conjugate group does not comprise a cleavable moiety.

In certain embodiments, modified crRNAs comprise a modified oligonucleotide. In certain embodiments, modified crRNAs consist of a modified oligonucleotide. Modified oligonucleotides described herein are suitable for use as crRNA.

In certain embodiments, modified crRNAs comprise at least three of the following features:
a. two linker nucleosides linked to the 5′-end of the CRISPR recognition portion of the modified crRNA;
b. 1^{st}, 8^{th}, and/or 9^{th} nucleoside from the 5′-end of the target recognition portion of the modified crRNA independently comprising 2′-F or 2′-H(H) modified sugar moiety;
c. at least one terminal phosphorothioate internucleoside linkage at each of the 3′ and 5′ termini of the modified crRNA
d. each nucleoside of the CRISPR recognition portion comprising an unmodified sugar moiety
e. one to five 3′-terminal nucleosides of the modified crRNA comprising independently selected modified sugar moieities
   In certain such embodiments, the modified crRNA comprises features (b), (d), and (e). In certain embodiments, the modified crRNA comprises features (b), (c), and (e). In certain embodiments, the modified crRNA comprises features (b), (c), and (d). In certain embodiments, the modified crRNA comprises features (b), (c), (d) and (e). In certain embodiments, the modified crRNA comprises features (a), (b), and (e). In certain embodiments, the modified crRNA comprises features (a), (c), and (e). In certain embodiments, the modified crRNA comprises features (a), (b), and (d). In certain embodiments, the modified crRNA comprises features (a), (b), (d) and (e). In certain embodiments, the modified crRNA comprises features (a), (b), (c) and (e). In certain embodiments, the modified crRNA comprises features (a), (b), (c), (d), and (e).

In certain embodiments, modified crRNAs comprise at least three of the following features:
a. two linker nucleosides linked to the 5′-end of the CRISPR recognition portion of the modified crRNA;
b. 1^{st}, 8^{th} and/or 9^{th} nucleoside from the 5′-end of the target recognition portion of the modified crRNA independently comprising 2′-F or 2′-H(H) modified sugar moiety;
c. at least one terminal phosphorothioate internucleoside linkage at each of the 3′ and 5′ termini of the modified crRNA
d. at least one nucleoside at position 5, 6, 7, 8, 11, or 12 from the 3′-end of the CRISPR recognition portion comprises a modified sugar moiety
e. one to five 3′-terminal nucleosides of the modified crRNA comprising independently selected modified sugar moieities
   In certain such embodiments, the modified crRNA comprises features (b), (d), and (e). In certain embodiments, the modified crRNA comprises features (b), (c), and (e). In certain embodiments, the modified crRNA comprises features (b), (c), and (d). In certain embodiments, the modified crRNA comprises features (b), (c), (d) and (e). In certain embodiments, the modified crRNA comprises features (a), (b), and (e). In certain embodiments, the modified crRNA comprises features (a), (c), and (e). In certain embodiments, the modified crRNA comprises features (a), (b), and (d). In certain embodiments, the modified crRNA comprises features (a), (b), (d) and (e). In certain embodiments, the modified crRNA comprises features (a), (b), (c) and (e). In certain embodiments, the modified crRNA comprises features (a), (b), (c), (d), and (e).

In certain embodiments, the modified crRNA comprises any combination of features (a), (b), (c), (d), and (e) listed in the table below, wherein one selection is made for each of (a), (b), (c), (d), and (e).

TABLE B
Features of certain modified crRNAs

| | | (c) terminal | | (e) sugar |
|---|---|---|---|---|
| | (b) modifications | phosphorothioate | (d) sugar moieties | modifications of |
| (a) linker | of target | (PS)internucleoside | of CRISPR | 3′-terminal |
| nucleosides | recognition portion | linkages | recognition portion | nucleosides |
| Two modified | 2′-H(H) modified | Two PS linkages at | Each nucleoside of | The two 3′-terminal |
| linker nucleosides | nucleosides at | each terminus | CRISPR recognition | nucleosides |
| | positions 1, 8, and 9 | | portion comprises | comprise modified |
| | | | an unmodified sugar | sugar moieties |
| | | | moiety | |
| Two 2′-H(H) | 2′-H(H) modified | Two PS linkages at | N/A (at least one | The five 3′-terminal |
| modified linker | nucleosides at | 5′ terminus and six | nucleoside of | nucleosides |
| nucleosides | positions 1 and 8 | PS linakges at 3′- | CRISPR recognition | comprise modified |
| | | terminus | portion comprises a | sugar moieties |
| | | | modified sugar) | |
| Two 2′-OMe | 2′-H(H) modified | N/A (at least one | bicyclic modified | The two 3′-terminal |
| modified | linker | nucleosides at | terminus has a non- | nucleoside at nucleosides |
| nucleosides | positions 1, and 9 | PS terminal linkage) | position 11 from the | comprise 2′-OMe |
| | | | 3′-end of the | modified sugar |
| | | | CRISPR recognition | moieties |
| | | | portion | |
| Two cEt modified | 2′-H(H) modified | | bicyclic modified | The two 3′-terminal |
| linker nucleosides | nucleosides at | | nucleoside at | nucleosides |
| | positions 8, and 9 | | position 12 from the | comprise cEt |
| | | | 3′-end of the | modified sugar |
| | | | CRISPR recognition | moieties |
| | | | portion | |
| One modified linker | 2′-H(H) modified | | bicyclic modified | The five 3′-terminal |
| nucleoside and one | nucleoside at | | nucleosides at | nucleosides |
| unmodified linker | position 1 | | positions 11 and 12 | comprise 2′-0Me |
| nucleoside | | | from | the 3′-end of |
| | | | the CRISPR | modified sugar |
| | | | recognition portion | moieties |
| One 2′-OMe | N/A (No modified | | | The five 3′-terminal |
| modified linker | nucleosides at any | | | nucleosides |
| nucleoside and one | of positions 1, 8, or | | | comprise 2′-F |
| unmodified linker | 9) | | | modified sugar |
| nucleoside | | | | moieties |
| One cEt modified | 2′-H(H) modified | | | N/A (The 3′- |
| linker nucleoside | nucleosides at | | | terminal nucleoside |
| and one unmodified | positions 14, 15, | | | comprises an |
| linker nucleoside | and 16 | | | unmodified sugar |
| | | | | moiety.) |
| N/A (zero or one | 2′-H(H) modified | | | |
| linker nucleosides) | nucleosides at | | | |
| | positions 14, 15, 16, | | | |
| | and at least one of | | | |
| | 1, 8, and 9 | | | |
| | 2′-F modified | | | |
| | nucleosides at | | | |
| | positions 1, 8, | | | |
| | and/or 9 | | | |
| | 2′-F modified | | | |
| | nucleosides at | | | |
| | positions 14, 15, | | | |
| | and/or 16 | | | |

Certain modified oligonucleotides have one or more asymmetric center and thus give rise to enantiomers, diastereomers, and other stereoisomeric configurations that may be defined, in terms of absolute stereochemistry, as (R) or (S), as a or β such as for sugar anomers, or as (D) or (L) such as for amino acids etc. Included in the modified oligonucleotides provided herein are all such possible isomers, including their racemic and optically pure forms, unless specified otherwise. Likewise, all cis- and trans-isomers and tautomeric forms are also included.

In certain embodiments, such modified oligonucleotides may contain any combination of the modified sugar moieites, modified nucleobases, modified internucleoside linkages, motifs, and/or lengths described herein.

Certain Methods of Use Comprising Modified crRNA

In certain embodiments, methods comprising contacting a cell with a compound comprising a modified crRNA are in vitro methods. In certain embodiments, methods comprising contacting a cell with a compound comprising a modified crRNA are ex vivo methods. In certain embodiments, methods comprising contacting a cell with a compound comprising a modified crRNA are in vivo methods.

Various CRISPR nuclease variants, both naturally occurring and genetically engineered, can be used in the methods of the present invention. Such variants include but are not limited to inactive nuclease mutants that are used in applications that do not require target nucleic acid cleavage, such as gene activation; and truncated nuclease variants that are suitable for expression in certain vectors, such as AAV vectors. In certain such embodiments, the CRISPR nuclease variant is a Cpf1 nuclease variant.

In certain embodiments, methods comprising contacting a cell with a compound comprising a modified crRNA further comprise contacting the cell with a second compound to inhibit (or turn off) the CRISPR system after the target gene is edited.

In certain embodiments, gene editing methods comprising contacting a cell with a compound comprising a modified crRNA produce fewer and/or less deleterious off-target effects than gene editing methods that use of an unmodified crRNA in place of the modified crRNAs of the invention.

The disclosure includes the following numbered embodiments:

### Embodiment 1

A compound comprising a modified crRNA consisting of 35-45 linked nucleosides.

### Embodiment 2

A compound comprising a modified crRNA, wherein the CRISPR recognition portion of the modified crRNA consists of 17-20 linked nucleosides.

### Embodiment 3

A compound comprising a modified crRNA, wherein the target recognition portion of the modified crRNA consists of 18-23 linked nucleosides.

### Embodiment 4

A compound comprising a modified crRNA, wherein the modified crRNA comprises at least one linker nucleoside.

### Embodiment 5

A compound comprising a 5′-stabilized modified crRNA.

### Embodiment 6

The compound of any of embodiments 1-5, wherein the compound comprises a stabilizing conjugate group.

### Embodiment 7

The compound of any of embodiments 1-5, wherein the crRNA comprises at least one linker nucleoside comprising a stabilizing modification.

### Embodiment 8

The compound of any of embodiments 1-4, wherein the modified crRNA is 5′-stabilized.

### Embodiment 9

The compound of any of embodiments 1-8, wherein the modified crRNA is 3′-stabilized.

### Embodiment 10

The compound of any of embodiments 1-9, wherein the CRISPR recognition portion of the modified crRNA binds to a Cpf1 nuclease.

### Embodiment 11

The compound of any of embodiments 1-10, wherein the target recognition portion of the modified crRNA comprises at least one modification that increases affinity of the crRNA for a target DNA or RNA.

### Embodiment 12

The compound of any of embodiments 10-11, wherein the CRISPR recognition portion of the modified crRNA comprises at least one modification that increases affinity of the crRNA for a Cpf1 nuclease.

### Embodiment 13

The compound of any of embodiments 1-12, wherein at least one nucleobase of the modified crRNA is thymine.

### Embodiment 14

The compound of any of embodiments 1-13, wherein at least one nucleobase of the modified crRNA is a modified nucleobase.

### Embodiment 15

The compound of embodiment 14, wherein the modified nucleobase is 5-methyl cytosine.

### Embodiment 16

The compound of any of embodiments 1-15, wherein modified crRNA consists of 35-42 linked nucleosides.

### Embodiment 17

The compound of any of claim 1-15, wherein the modified crRNA consists of 36-40 linked nucleosides.

### Embodiment 18

The compound of any of embodiments 1-17, wherein the modified crRNA comprises at least two linker nucleosides.

### Embodiment 19

The compound of embodiment 18, wherein at least two linker nucleosides are linked to the CRISPR recognition portion of the modified crRNA.

### Embodiment 20

The compound of embodiment 19, wherein at least two linker nucleosides are linked to the 5′-end of the CRISPR recognition portion of the modified crRNA.

### Embodiment 21

The compound of any of embodiments 1-20, wherein the CRISPR recognition portion of the modified crRNA consists of 18-20 linked nucleosides.

### Embodiment 22

The compound of embodiment 21, wherein the CRISPR recognition portion of the modified crRNA consists of 18 linked nucleosides.

### Embodiment 23

The compound of embodiment 21, wherein the CRISPR recognition portion of the modified crRNA consists of 19 linked nucleosides.

### Embodiment 24

The compound of embodiment 21, wherein the CRISPR recognition portion of the modified crRNA consists of 20 linked nucleosides.

### Embodiment 25

The compound of any of embodiments 1-24, wherein the target recognition protion of the modified crRNA consists of 18-22 linked nucleosides.

### Embodiment 26

The compound of any of embodiments 1-24, wherein the target recognition protion of the modified crRNA consists of 18-20 linked nucleosides.

### Embodiment 27

The compound of embodiment 26, wherein the target recognition protion of the modified crRNA consists of 18 linked nucleosides.

### Embodiment 28

The compound of embodiment 26, wherein the target recognition protion of the modified crRNA consists of 19 linked nucleosides.

### Embodiment 29

The compound of embodiment 26, wherein the target recognition protion of the modified crRNA consists of 20 linked nucleosides.

### Embodiment 30

The compound of any of embodiments 1-29, wherein at least one internucleoside linkage of the modified crRNA is a modified internucleoside linkage.

### Embodiment 31

The compound of embodiment 30, wherein at least one internucleoside linkage is a phosphorothioate internucleoside linkage.

### Embodiment 32

The compound of embodiment 30 or 31, wherein each internucleoside linkage of the modified crRNA is a modified internucleoside linkage.

### Embodiment 33

The compound of any of embodiments 30-32, wherein at least one internucleoside linkage is a neutral internucleoside linkage.

### Embodiment 34

The compound of embodiment 33, wherein at least one modified internucleoside linkage comprises a methoxypropyl group.

### Embodiment 35

The compound of embodiment 33, wherein at least one modified internucleoside linkage comprises a phosphonoacetate.

### Embodiment 36

The compound of embodiment 33, wherein at least one modified internucleoside linkage comprises a methylphosphonate.

### Embodiment 37

The compound of any of embodiments 1-31, wherein each internucleoside linkage of the modified crRNA is a phosphodiester internucleoside linkage or a phosphorothioate internucleoside linkage.

### Embodiment 38

The compound of any of embodiments 30, 31, or 33-37, wherein at least two internucleoside linkages of the modified crRNA are modified internucleoside linkages.

### Embodiment 39

The compound of embodiment 38, wherein at least two modified internucleoside linkages of the modified crRNA are the same as one another.

### Embodiment 40

The compound of any of embodiments 1-39, wherein the modified crRNA comprises one to five contiguous phosphorothioate internucleoside linkages at the 5′-end of the modified crRNA.

### Embodiment 41

The compound of embodiment 40, wherein the modified crRNA comprises one phosphorothioate internucleoside linkage at the 5′-end of the modified crRNA.

### Embodiment 42

The compound of embodiment 40, wherein the modified crRNA comprises two contiguous phosphorothioate internucleoside linkages at the 5′-end of the modified crRNA.

### Embodiment 43

The compound of any of embodiments 1-42, wherein the modified crRNA comprises at least one linker nucleoside that is linked to the CRISPR recognition portion of the modified crRNA by a modified internucleoside linkage.

### Embodiment 44

The compound of embodiment 43, wherein the modified internucleoside linkage that links the at least one linker nucleoside to the CRISPR recognition portion of the modified crRNA is a phosphorothiaote internucleoside linkage.

### Embodiment 45

The compound of embodiment 44, wherein the modified crRNA comprises two linker nucleosides.

### Embodiment 46

The compound of embodiment 45, wherein the linker nucleosides are linked to each other by a modified internucleoside linkage.

### Embodiment 47

The compound of embodiment 46, wherein the modified internucleoside that links the linker nucleosides to each other is a phosphorothioate internucleoside linkage.

### Embodiment 48

The compound of any embodiments 43-44, wherein the modified crRNA comprises more than two linker nucleosides.

### Embodiment 49

The compound of any of embodiments 1-48, wherein the modified crRNA comprises one to six modified internucleoside linkages within the target recognition portion of the modified crRNA.

### Embodiment 50

The compound of embodiment 49, wherein the one to six modified internucleoside linkages within the target recognition portion of the modified crRNA are contiguous.

### Embodiment 51

The compound of embodiment 49, wherein the one to six modified internucleoside linkages within the target recognition portion of the modified crRNA alternate with unmodified internucleoside linkages.

### Embodiment 52

The compound of any of embodiments 49-51, wherein the 3′-end of the target recognition portion of the modified crRNA contains the one to six modified internucleoside linkages.

### Embodiment 53

The compound of any of embodiments 50-52, wherein the target recognition portion of the modified crRNA comprises one modified internucleoside linkage.

### Embodiment 54

The compound of any of embodiments 50-52, wherein the target recognition portion of the modified crRNA comprises two modified internucleoside linkages.

### Embodiment 55

The compound of any of embodiments 50-52, wherein the target recognition portion of the modified crRNA comprises three modified internucleoside linkages.

### Embodiment 56

The compound of any of embodiments 50-52, wherein the target recognition portion of the modified crRNA comprises four modified internucleoside linkages.

### Embodiment 57

The compound of any of embodiments 50-52, wherein the target recognition portion of the modified crRNA comprises five modified internucleoside linkages.

### Embodiment 58

The compound of any of embodiments 50-52, wherein the target recognition portion of the modified crRNA comprises six modified internucleoside linkages.

### Embodiment 59

The compound of any of embodiments 49-58, wherein at least one internucleoside linkage within the target recognition portion of the modified crRNA is a phosphorothioate internucleoside linkage.

### Embodiment 60

The compound of any of embodiments 49-58, wherein all of the modified internucleoside linkages within the target recognition portion of the modified crRNA are phosphorothioate internucleoside linkages.

### Embodiment 61

The compound of any of embodiments 1-60, wherein the target recognition portion of the modified crRNA is directly or indirectly linked to the 3′ end of the CRISPR recognition portion of the modified crRNA.

### Embodiment 62

The compound of any of embodiments 1-61, wherein at least one nucleoside of the modified crRNA comprises a modified sugar moiety.

### Embodiment 63

The compound of embodiment 62, wherein the 5′-terminal nucleoside of the crRNA comprises a modified sugar moiety.

### Embodiment 64

The compound of embodiment 63, wherein the 5′-terminal nucleoside comprises a linearly modified sugar moiety.

### Embodiment 65

The compound of embodiment 64, wherein the 5′-terminal nucleoside comprises a 2′-modified sugar moiety.

### Embodiment 66

The compound of embodiment 63, wherein the 5′-terminal nucleoside comprises a bicyclic sugar moiety.

### Embodiment 67

The compound of embodiment 63, wherein the 5′-terminal nucleoside comprises a modified sugar moiety selected from among: 2′-O-methyl, 2′-MOE, 2′-F, cEt, and LNA.

### Embodiment 68

The compound of any of embodiments 1-67, wherein the 5′-terminal nucleoside is a linker nucleoside.

### Embodiment 69

The compound of any of embodiments 62-68, wherein the 5^{th} nucleoside from the 5′-end of the CRISPR recognition portion comprises a modified sugar moiety.

### Embodiment 70

The compound of any of embodiments 62-69, wherein the 6^{th} nucleoside from the 5′-end of the CRISPR recognition portion comprises a modified sugar moiety.

### Embodiment 71

The compound of any of embodiments 62-70, wherein the 7^{th} nucleoside from the 5′-end of the CRISPR recognition portion comprises a modified sugar moiety.

### Embodiment 72

The compound of any of embodiments 62-71, wherein the 10^{th} nucleoside from the 5′-end of the CRISPR recognition portion comprises a modified sugar moiety.

### Embodiment 73

The compound of any of embodiments 62-72, wherein the 14^{th} nucleoside from the 5′-end of the CRISPR recognition portion comprises a modified sugar moiety.

### Embodiment 74

The compound of any of embodiments 62-73, wherein the 1st nucleoside from the 3′-end of the CRISPR recognition portion comprises a modified sugar moiety.

### Embodiment 75

The compound of any of embodiments 69-74, wherein at least one modified sugar moiety selected from among: 2′-O-methyl, 2′-MOE, 2′-F, cEt, and LNA.

### Embodiment 76

The compound of any of embodiments 69-74, wherein each modified sugar moiety is independently selected from among: 2′-O-methyl, 2′-MOE, 2′-F, cEt, and LNA.

### Embodiment 77

The compound of embodiment 62, wherein the 3′-terminal nucleoside of the modified crRNA comprises a modified sugar moiety.

### Embodiment 78

The compound of embodiment 77, wherein the 3′-terminal nucleoside comprises a linearly modified sugar moiety.

### Embodiment 79

The compound of embodiment 78, wherein the 3′-terminal nucleoside comprises a 2′-modified sugar moiety.

### Embodiment 80

The compound of embodiment 77, wherein the 3′-terminal nucleoside comprises a bicyclic sugar moiety.

### Embodiment 81

The compound of embodiment 77, wherein the 3′-terminal nucleoside comprises a modified sugar moiety selected from among: 2′-O-methyl, 2′-MOE, 2′-F, cEt, and LNA.

### Embodiment 82

The compound of any of embodiments 62-81, wherein the 1st nucleoside from the 5′-end of the target recognition portion comprises a modified sugar moiety.

### Embodiment 83

The compound of any of embodiments 62-82, wherein the 8th nucleoside from the 5′-end of the target recognition portion comprises a modified sugar moiety.

### Embodiment 84

The compound of any of embodiments 62-83, wherein the 9th nucleoside from the 5′-end of the target recognition portion comprises a modified sugar moiety.

### Embodiment 85

The compound of any of embodiments 62-84, wherein one to five 3′-terminal nucleosides of the target recognition portion of the modified crRNA each comprise a modified sugar moiety.

### Embodiment 86

The compound of embodiment 85, wherein the one to five 3′-terminal nucleosides of the target recognition portion of the modified crRNA each comprise the same modified sugar moiety.

### Embodiment 87

The compound of embodiment 84 or 85, wherein the modified sugar moieties of the one to five 3′-terminal nucleosides of the target recognition portion are each independently selected from among 2′-O-methyl, 2′-MOE, 2′-F, cEt, and LNA.

### Embodiment 88

The compound of any of embodiments 1-87, wherein the target recognition portion of the modified crRNA comprises at least one unmodified sugar moiety.

### Embodiment 89

The compound of any of embodiments 1-88, wherein the CRISPR recognition portion of the modified crRNA comprises at least one unmodified sugar moiety.

### Embodiment 90

The compound of any of embodiments 1-89, wherein the modified crRNA comprises at least one linker nucleoside that comprises an unmodified sugar moiety.

### Embodiment 91

The compound of any of embodiments 1-90, wherein the compound consists of the modified crRNA.

### Embodiment 92

The compound of any of embodiments 1-91, wherein the nucleobase sequence of the target recognition portion of the modified crRNA is at least 90% complementary to a target DNA or RNA.

### Embodiment 93

The compound of embodiment 92, wherein the nucleobase sequence of the target recognition portion of the modified crRNA is 100% complementary to a target DNA or RNA.

### Embodiment 94

The compound of any of embodiments 1-93, wherein the modified crRNA comprises a self-complementary region.

### Embodiment 95

The compound of embodiment 94, wherein the self-complementary region is within the CRISPR recognition portion of the modified crRNA.

### Embodiment 96

The compound of embodiment 94 or 95, wherein the self-complementary region can form a hairpin.

### Embodiment 97

The compound of any of embodiments 94-96, wherein the self-complementary region comprises at least one modification that increases the stability of the self-complementary region.

### Embodiment 98

The compound of any of embodiments 94-97, wherein the self-complementary region comprises at least one modification that increases the hybridization affinity of the self-complementary region.

### Embodiment 99

The compound of any of embodiments 1-98, wherein the nucleobase sequence of the CRISPR recognition portion of the modified crRNA comprises at least 12 contiguous nucleobases of a sequence selected from Table A.

### Embodiment 100

The compound of any of embodiments 1-98, wherein the nucleobase sequence of the CRISPR recognition portion of the modified crRNA consists of a sequence or a portion of a sequence selected from Table A.

### Embodiment 101

The compound of any of embodiments 1-100, wherein the nucleobase sequence of the CRISPR recognition portion of the modified crRNA comprises the sequence XCXACX, wherein each X is, independently, a U nucleobase or a T nucleobase.

### Embodiment 102

The compound of any of embodiments 1-100, wherein the nucleobase sequence of the CRISPR recognition portion of the modified crRNA comprises the sequence GXAGAX, wherein each X is, independently, a U nucleobase or a T nucleobase.

### Embodiment 103

The compound of any of embodiments 1-100, wherein the nucleobase sequence of the CRISPR recognition portion of the modified crRNA comprises the sequence XCXACX and the sequence GXAGAX, wherein each X is, independently, a U nucleobase or a T nucleobase.

### Embodiment 104

The compound of any of embodiments 1-90 or 92-103, wherein the compound comprises a conjugate group.

### Embodiment 105

The compound of embodiment 104, wherein the conjugate group comprises GalNAc.

### Embodiment 106

The compound of embodiment 104, wherein the conjugate group is lipophilic.

### Embodiment 107

The compound of embodiment 106, wherein the conjugate group comprises a lipid.

### Embodiment 108

A pharmaceutical composition comprising the compound of any of embodiments 1-107.

### Embodiment 109

A method comprising contacting a cell with the compound or composition of any of embodiments 1-108.

### Embodiment 110

The method of embodiment 109, wherein the cell expresses a Cpf1 nuclease.

### Embodiment 111

A method comprising contacting a cell with the compound or composition of any of embodiments 1-108 and a plasmid that encodes a Cpf1 nuclease.

### Embodiment 112

A method comprising contacting a cell with the compound or composition of any of embodiments 1-108 and an mRNA that encodes a Cpf1 nuclease.

### Embodiment 113

The method of any of embodiments 109-112, wherein the modified crRNA is taken up by the cell in the absence of a transfection reagent.

### Embodiment 114

The method of any of embodiments 109-113, wherein the cell is in an animal.

### Embodiment 115

A method comprising administering to an animal the compound or composition of any of embodiments 1-108.

### Embodiment 116

The method of embodiment 115, wherein the administration is subcutaneous.

### Embodiment 117

The method of embodiment 115, wherein the administration is intrathecal.

### Embodiment 118

The method of any of embodiments 115-117 comprising administering a plasmid that encodes a Cpf1 nuclease.

### Embodiment 119

The method of any of embodiments 115-117 wherein the animal expresses a Cpf1 nuclease.

### Embodiment 120

The method of embodiment 111 or 118, wherein the plasmid is delivered to cells within the animal via an adeno-associated virus (AAV).

### Embodiment 121

The method of embodiment 111 or 118, wherein the plasmid is delivered to cells within the animal via a lentivirus.

### Embodiment 122

The method of any of embodiments 109-121, wherein a target gene is edited.

### Embodiment 123

The method of embodiment 122, wherein the modified crRNA is degraded in a cell after the target gene is edited in the cell.

### Embodiment 124

The method of any of embodiments 110-112 or 118-123, wherein the Cpf1 nuclease does not exhibit nuclease activity in the absence of the modified crRNA.

### Embodiment 125

The method of any of embodiments 109-124 comprising contacting the cell with a second compound that degrades or inhibits the activity or expression of the modified crRNA or a Cpf1 nuclease.

### Embodiment 126

The method of embodiment 125, wherein the cell is contacted with the second compound after a target gene has been edited.

### Embodiment 127

The method of embodiment 125 or 126, wherein the second compound comprises an oligonucleotide that is complementary to the modified crRNA.

### Embodiment 128

The method of embodiment 125 or 126, wherein the second compound comprises a crRNA that targets a Cpf1 nuclease gene.

### Embodiment 129

The method of embodiment 125 or 126, wherein the second compound comprises an oligonucleotide that is complementary to a Cpf1 transcript.

### Embodiment 130

The method of embodiment 128 or 129, wherein the expression of the Cpf1 nuclease is inhibited.

### Embodiment 131

The method of any of embodiments 114-130, wherein the animal is a human.

### Embodiment 132

The method of any of embodiments 109-131, wherein editing of at least one off-target gene is reduced relative to editing the at least one off-target gene when unmodified crRNA or a compound comprising more than 45 nucleosides is used in place of the modified crRNA.

### Embodiment 133

The method of any of embodiments 115 or 118-132, wherein the administration is intravitreal.

### Embodiment 134

The method of any of embodiments 109-113, wherein the cell is a plant cell.

### Embodiment 135

The method of any of embodiments 109-114, wherein the cell is a T-cell.

### Embodiment 136

A method of treating a disease in an individual comprising administering the compound of any of embodiments 1-107 or the composition of embodiment 108 to the individual, thereby treating the disease in the individual.

### Embodiment 137

Use of the compound of any of embodiments 1-107 or the composition of embodiment 108 for the treatment of a disease.

### Embodiment 138

Use of the compound of any of embodiments 1-107 or the composition of embodiment 108 for preparation of a medicament.

### Embodiment 139

A method of administering the compound of any of embodiments 1-107 or the composition of embodiment 108 to an animal, and harvesting an organ from the animal for transplantation into a human.

### Embodiment 140

The compound of any of embodiments 90-107, wherein the 5′-terminal nucleoside comprises a cEt modified sugar moiety.

### Embodiment 141

The compound of embodiment 140, wherein the 3′-end of the target recognition portion of the modified crRNA contains two contiguous phosphorothioate internucleoside linkages.

### Embodiment 142

The compound of any of embodiments 140-141, wherein each internucleoside linkage of the CRISPR recognition portion of the modified crRNA is phosphorothioate.

### Embodiment 143

The compound of any of embodiments 140-142, wherein the two 3′-terminal nucleosides of the target recognition portion of the modified crRNA each comprise a 2′-O-methyl modified sugar moiety.

### Embodiment 144

The compound of any of embodiments 140-143, wherein the 1″ nucleoside from the 5′-end of the CRISPR recognition portion comprises an unmodified sugar moiety.

### Embodiment 145

The compound of any of embodiments 140-144, wherein the modified crRNA comprises 30-38 unmodified sugar moieties.

### Embodiment 146

The compound of embodiment 145, wherein the modified crRNA comprises 36 unmodified sugar moieties.

### Embodiment 147

A pharmaceutical composition comprising the compound of any of embodiments 140-146.

### Embodiment 148

A method comprising contacting a cell with the compound or composition of any of embodiments 140-147.

### Embodiment 149

The method of embodiment 148, wherein the cell expresses a Cpf1 nuclease.

### Embodiment 150

A method comprising contacting a cell with the compound or composition of any of embodiments 140-147 and a plasmid that encodes a Cpf1 nuclease.

### Embodiment 151

A method comprising contacting a cell with the compound or composition of any of embodiments 140-147 and an mRNA that encodes a Cpf1 nuclease.

### Embodiment 152

The method of any of embodiments 148-151, wherein the modified crRNA is taken up by the cell in the absence of a transfection reagent.

### Embodiment 153

The method of any of embodiments 148-152, wherein the cell is in an animal.

### Embodiment 154

A method comprising administering to an animal the compound or composition of any of embodiments 140-147.

### Embodiment 155

The method of embodiment 154, wherein the administration is subcutaneous.

### Embodiment 156

The method of embodiment 154, wherein the administration is intrathecal.

### Embodiment 157

The method of any of embodiments 154-156 comprising administering a plasmid that encodes a Cpf1 nuclease.

### Embodiment 158

The method of any of embodiments 154-156 wherein the animal expresses a Cpf1 nuclease.

### Embodiment 159

The method of embodiment 150 or 157, wherein the plasmid is delivered to cells within the animal via an adeno-associated virus (AAV).

### Embodiment 160

The method of embodiment 150 or 157, wherein the plasmid is delivered to cells within the animal via a lentivirus.

### Embodiment 161

The method of any of embodiments 148-160, wherein a target gene is edited.

### Embodiment 162

The method of embodiment 161, wherein the modified crRNA is degraded in a cell after the target gene is edited in the cell.

### Embodiment 163

The method of any of embodiments 149-151 or 157-162, wherein the Cpf1 nuclease does not exhibit nuclease activity in the absence of the modified crRNA.

### Embodiment 164

The method of any of embodiments 148-163 comprising contacting the cell with a second compound that degrades or inhibits the activity or expression of the modified crRNA or a Cpf1 nuclease.

### Embodiment 165

The method of embodiment 164, wherein the cell is contacted with the second compound after a target gene has been edited.

### Embodiment 166

The method of embodiment 164 or 165, wherein the second compound comprises an oligonucleotide that is complementary to the modified crRNA.

### Embodiment 167

The method of embodiment 164 or 165, wherein the second compound comprises a crRNA that targets a Cpf1 nuclease gene.

### Embodiment 168

The method of embodiment 164 or 165, wherein the second compound comprises an oligonucleotide that is complementary to a Cpf1 transcript.

### Embodiment 169

The method of embodiment 167 or 168, wherein the expression of the Cpf1 nuclease is inhibited.

### Embodiment 170

The method of any of embodiments 153-169, wherein the animal is a human.

### Embodiment 171

The method of any of embodiments 148-170, wherein editing of at least one off-target gene is reduced relative to editing the at least one off-target gene when unmodified crRNA or a compound comprising more than 45 nucleosides is used in place of the modified crRNA.

### Embodiment 172

The method of any of embodiments 154 or 157-171, wherein the administration is intravitreal.

### Embodiment 173

The method of any of embodiments 148-152, wherein the cell is a plant cell.

### Embodiment 174

The method of any of embodiments 148-153, wherein the cell is a T-cell.

### Embodiment 175

A method of treating a disease in an individual comprising administering the compound of any of embodiments 140-146 or the composition of embodiment 147 to the individual.

### Embodiment 176

A method of treating a disease in an individual comprising administering the compound of any of embodiments 140-146 or the composition of embodiment 147 to the individual, thereby treating the disease in the individual.

### Embodiment 177

Use of the compound of any of embodiments 140-146 or the composition of embodiment 147 for the treatment of a disease.

### Embodiment 178

Use of the compound of any of embodiments 140-146 or the composition of embodiment 147 for preparation of a medicament.

### Embodiment 179

A method of administering the compound of any of embodiments 140-146 or the composition of embodiment 147 to an animal, and harvesting an organ from the animal for transplantation into a human.

### Embodiment 180

The compound of any of embodiments 91-107 or 140-146, wherein at least one modified nucleoside of the modified crRNA is a 2′-deoxynucleoside.

### Embodiment 181

The compound of any of embodiments 91-107 or 140-146, wherein at least one modified nucleoside of the modified crRNA comprises a linearly modified sugar moiety having a 2′-H substitution.

### Embodiment 182

The compound of any of embodiments 91-107 or 140-146, wherein at least one modified nucleoside of the modified crRNA comprises a modified 2′-H(H) sugar moiety as found in naturally occurring DNA.

### Embodiment 183

The compound of any of embodiments 91-107, 140-146, or 180-182, wherein the modified crRNA consists of 40 linked nucleosides.

### Embodiment 184

The compound of any of embodiments 91-107, 140-146, or 180-182, wherein the modified crRNA consists of 43 linked nucleosides.

### Embodiment 185

The compound of any of embodiments 91-107, 140-146, or 180-182, wherein the modified crRNA consists of 45 linked nucleosides.

### Embodiment 186

The compound of any of embodiments 91-107, 140-146, or 180-185, wherein the target recognition portion of the modified crRNA is at least 90% complementary to a DNMT1 nucleic acid.

### Embodiment 187

The compound of embodiment 186, wherein the target recognition portion is 100% complementary to a DNMT1 nucleic acid.

### Embodiment 188

The compound of embodiment 186 or 187, wherein the DNMT1 nucleic acid is a deoxyribonucleic acid.

### Embodiment 189

The compound of embodiment 188, wherein the DNMT1 nucleic acid is a human deoxyribonucleic acid.

### Embodiment 190

The compound of any of embodiments 91-107, 140-146, or 180-185, wherein the target recognition portion of the modified crRNA is at least 90% complementary to a LDLR nucleic acid.

### Embodiment 191

The compound of embodiment 190, wherein the target recognition portion is 100% complementary to a LDLR nucleic acid. The compound of embodiment 190 or 191, wherein the LDLR nucleic acid is a deoxyribonucleic acid.

### Embodiment 192

The compound of embodiment 191, wherein the LDLR nucleic acid is a human deoxyribonucleic acid.

### Embodiment 193

The compound of any of embodiments 91-107, 140-146, or 180-192, wherein the two 3′-terminal nucleosides of the modified crRNA comprise independently selected modified sugar moieities.

### Embodiment 194

The compound of any of embodiments 91-107, 140-146, or 180-192, wherein the three 3′-terminal nucleosides of the modified crRNA comprise independently selected modified sugar moieities.

### Embodiment 195

The compound of any of embodiments 91-107, 140-146, or 180-192, wherein the four 3′-terminal nucleosides of the modified crRNA comprise independently selected modified sugar moieities.

### Embodiment 196

The compound of any of embodiments 91-107, 140-146, or 180-192, wherein the five 3′-terminal nucleosides of the modified crRNA comprise independently selected modified sugar moieities.

### Embodiment 197

The compound of any of embodiments 77 or 193-196, wherein the modified sugar moieties of the 3′-terminal modified nucleosides are selected from among 2′-H(H), 2′-O-methyl, 2′-F, cEt, and LNA modified sugar moieties.

### Embodiment 198

The compound of embodiment 197, wherein the modified sugar moieties of the 3′-terminal modified nucleosides are selected from among 2′-H(H), 2′-O-methyl, and cEt modified sugar moieties.

### Embodiment 199

The compound of embodiment 197, wherein the modified sugar moieties of the 3′-terminal modified nucleosides are selected from among 2′-H(H) and 2′-O-methyl modified sugar moieties.

### Embodiment 200

The compound of embodiment 197, wherein the modified sugar moieties of the 3′-terminal modified nucleosides are selected from among cEt and LNA modified sugar moieties.

### Embodiment 201

The compound of any of embodiments 82-107, 140-146, or 180-200, wherein the 1st nucleoside from the 5′-end of the target recognition portion comprises a 2′-H(H) or 2′-F modified sugar moiety.

### Embodiment 202

The compound of any of embodiments 82-107, 140-146, or 180-201, wherein the 8th nucleoside from the 5′-end of the target recognition portion comprises a 2′-H(H) or 2′-F modified sugar moiety.

### Embodiment 203

The compound of any of embodiments 82-107, 140-146, or 180-202, wherein the 9th nucleoside from the 5′-end of the target recognition portion comprises a 2′-H(H) or 2′-F modified sugar moiety.

### Embodiment 204

The compound of any of embodiments 91-107, 140-146, or 180-203, wherein the modified crRNA comprises at least three of the following features:
a. two linker nucleosides linked to the 5′-end of the CRISPR recognition portion of the modified crRNA;
b. 1^{st}, 8^{th}, and/or 9^{th} nucleoside from the 5′-end of the target recognition portion of the modified crRNA independently comprising 2′-F or 2′-H(H) modified sugar moiety;
c. at least one terminal phosphorothioate internucleoside linkage at each of the 3′ and 5′ termini of the modified crRNA
d. each nucleoside of the CRISPR recognition portion comprising an unmodified sugar moiety
e. one to five 3′-terminal nucleosides of the modified crRNA comprising independently selected modified sugar moieities

### Embodiment 205

The compound of any of embodiments 1-107, 140-146, or 180-204, wherein the modified crRNA is a salt.

### Embodiment 206

A pharmaceutical composition comprising the compound of any of embodiments 180-205.

### Embodiment 207

The pharmaceutical composition of any of embodiments 108, 147, or 206, wherein the pharmaceutical composition comprises a ribonucleoprotein complex.

### Embodiment 208

The pharmaceutical composition of embodiment 207, wherein the ribonucleoprotein complex comprises a Cpf1 nuclease and the compound comprising the modified crRNA.

### Embodiment 209

A method comprising contacting a cell with the compound or composition of any of embodiments 180-208.

### Embodiment 210

A method comprising contacting a cell with the compound or composition of any of embodiments 180-207, wherein the cell expresses a Cpf1 nuclease.

### Embodiment 211

A method comprising contacting a cell with the compound or composition of any of embodiments 180-207 and a plasmid that encodes a Cpf1 nuclease.

### Embodiment 212

A method comprising contacting a cell with the compound or composition of any of embodiments 180-207 and an mRNA that encodes a Cpf1 nuclease.

### Embodiment 213

The method of any of embodiments 209-212, wherein the modified crRNA is taken up by the cell in the absence of a transfection reagent.

### Embodiment 214

The method of any of embodiments 209-213, wherein the cell is in an animal.

### Embodiment 215

A method comprising administering to an animal the compound or composition of any of embodiments 180-208.

### Embodiment 216

The method of embodiment 215, wherein the administration is subcutaneous.

### Embodiment 217

The method of embodiment 215, wherein the administration is intrathecal.

### Embodiment 218

The method of any of embodiments 215-217 comprising administering a plasmid that encodes a Cpf1 nuclease.

### Embodiment 219

The method of any of embodiments 215-217 wherein the animal expresses a Cpf1 nuclease.

### Embodiment 220

The method of embodiment 211 or 218, wherein the plasmid is delivered to cells within the animal via an adeno-associated virus (AAV).

### Embodiment 221

The method of embodiment 211 or 218, wherein the plasmid is delivered to cells within the animal via a lentivirus.

### Embodiment 222

The method of any of embodiments 209-221, wherein a target gene is edited.

### Embodiment 223

The method of embodiment 222, wherein the modified crRNA is degraded in a cell after the target gene is edited in the cell.

### Embodiment 224

The method of any of embodiments 210-212 or 215-223, wherein the Cpf1 nuclease does not exhibit nuclease activity in the absence of the modified crRNA.

### Embodiment 225

The method of any of embodiments 209-224 comprising contacting the cell with a second compound that degrades or inhibits the activity or expression of the modified crRNA or a Cpf1 nuclease.

### Embodiment 226

The method of embodiment 225, wherein the cell is contacted with the second compound after a target gene has been edited.

### Embodiment 227

The method of embodiment 225 or 226, wherein the second compound comprises an oligonucleotide that is complementary to the modified crRNA.

### Embodiment 228

The method of embodiment 225 or 226, wherein the second compound comprises a crRNA that targets a Cpf1 nuclease gene.

### Embodiment 229

The method of embodiment 225 or 226, wherein the second compound comprises an oligonucleotide that is complementary to a Cpf1 transcript.

### Embodiment 230

The method of embodiment 228 or 229, wherein the expression of the Cpf1 nuclease is inhibited.

### Embodiment 231

The method of any of embodiments 214-230, wherein the animal is a human.

### Embodiment 232

The method of any of embodiments 209-231, wherein editing of at least one off-target gene is reduced relative to editing the at least one off-target gene when unmodified crRNA or a compound comprising more than 45 nucleosides is used in place of the modified crRNA.

### Embodiment 233

The method of any of embodiments 215 or 218-232, wherein the administration is intravitreal.

### Embodiment 234

The method of any of embodiments 209-213, wherein the cell is a plant cell.

### Embodiment 235

The method of any of embodiments 209-214, wherein the cell is a T-cell.

### Embodiment 236

A method of treating a disease in an individual comprising administering the compound of any of embodiments 180-205 or the composition of any of embodiments 206-208 to the individual.

### Embodiment 237

A method of treating a disease in an individual comprising administering the compound of any of embodiments 180-205 or the composition any of embodiments 206-208 to the individual, thereby treating the disease in the individual.

### Embodiment 238

Use of the compound of any of embodiments 180-205 or the composition of any of embodiments 206-208 for the treatment of a disease.

### Embodiment 239

Use of the compound of any of embodiments 180-205 or the composition of any of embodiments 206-208 for preparation of a medicament.

### Embodiment 240

A method of administering the compound of any of embodiments 180-205 or the composition of any of embodiments 206-208 to an animal, and harvesting an organ from the animal for transplantation into a human.

### Embodiment 241

The compound of any of embodiments 1-107, 140-146, or 180-205, wherein the CRISPR recognition portion of the modified crRNA comprises at least one modified sugar moiety.

### Embodiment 242

The compound of embodiment 241, wherein the at least one modified sugar moiety of the CRISPR recognition portion is a linearly modified sugar moiety.

### Embodiment 243

The compound of embodiment 241, wherein the at least one modified sugar moiety of the CRISPR recognition portion is a bicyclic sugar moiety.

### Embodiment 244

The compound of embodiment 243, wherein the bicyclic sugar moiety is cEt or LNA.

### Embodiment 245

The compound of embodiment 243, wherein the bicyclic sugar moiety is cEt.

### Embodiment 246

The compound of any of claims 241-245, wherein the 2nd nucleoside from the 3′-end of the CRISPR recognition portion comprises the at least one modified sugar moiety.

### Embodiment 247

The compound of any of claims 241-245, wherein the 3rd nucleoside from the 3′-end of the CRISPR recognition portion comprises the at least one modified sugar moiety.

### Embodiment 248

The compound of any of claims 241-245, wherein the 4th nucleoside from the 3′-end of the CRISPR recognition portion comprises the at least one modified sugar moiety.

### Embodiment 249

The compound of any of claims 241-245, wherein the 5th nucleoside from the 3′-end of the CRISPR recognition portion comprises the at least one modified sugar moiety.

### Embodiment 250

The compound of any of claims 241-245, wherein the 6th nucleoside from the 3′-end of the CRISPR recognition portion comprises the at least one modified sugar moiety.

### Embodiment 251

The compound of any of claims 241-245, wherein the 7th nucleoside from the 3′-end of the CRISPR recognition portion comprises the at least one modified sugar moiety.

### Embodiment 252

The compound of any of claims 241-245, wherein the 8th nucleoside from the 3′-end of the CRISPR recognition portion comprises the at least one modified sugar moiety.

### Embodiment 253

The compound of any of claims 241-245, wherein the 9th nucleoside from the 3′-end of the CRISPR recognition portion comprises the at least one modified sugar moiety.

### Embodiment 254

The compound of any of claims 241-245, wherein the 11th nucleoside from the 3′-end of the CRISPR recognition portion comprises the at least one modified sugar moiety.

### Embodiment 255

The compound of any of claims 241-245, wherein the 12th nucleoside from the 3′-end of the CRISPR recognition portion comprises the at least one modified sugar moiety.

### Embodiment 256

The compound of any of claims 241-245, wherein the 13th nucleoside from the 3′-end of the CRISPR recognition portion comprises the at least one modified sugar moiety.

### Embodiment 257

The compound of any of claims 241-245, wherein the 18^{th} nucleoside from the 3′-end of the CRISPR recognition portion comprises the at least one modified sugar moiety.

### Embodiment 258

The compound of any of claims 241-245, wherein the 11^{th} and 12^{th} nucleosides from the 3′-end of the CRISPR recognition portion each comprise a modified sugar moiety.

### Embodiment 259

The compound of any of claims 241-258, wherein the 1″ nucleoside from the 3′-end of the CRISPR recognition portion comprises an unmodified sugar moiety.

### Embodiment 260

The compound of any of claims 241-259, wherein the 10^{th} nucleoside from the 3′-end of the CRISPR recognition portion comprises an unmodified sugar moiety.

### Embodiment 261

The compound of any of claims 241-260, wherein the 14^{th} nucleoside from the 3′-end of the CRISPR recognition portion comprises an unmodified sugar moiety.

### Embodiment 262

The compound of any of claims 241-261, wherein the 15^{th} nucleoside from the 3′-end of the CRISPR recognition portion comprises an unmodified sugar moiety.

### Embodiment 263

The compound of any of claims 241-262, wherein the 16^{th} nucleoside from the 3′-end of the CRISPR recognition portion comprises an unmodified sugar moiety.

### Embodiment 264

The compound of any of claims 241-263, wherein the 17^{th} nucleoside from the 3′-end of the CRISPR recognition portion comprises an unmodified sugar moiety.

### Embodiment 265

The compound of any of claims 241-264, wherein the 1st nucleoside from the 5′-end of the CRISPR recognition portion comprises an unmodified sugar moiety.

### Embodiment 266

The compound of any of claims 241-265, wherein the 2nd nucleoside from the 5′-end of the CRISPR recognition portion comprises an unmodified sugar moiety.

### Embodiment 267

The compound of any of claims 241-266, wherein the 3rd nucleoside from the 5′-end of the CRISPR recognition portion comprises an unmodified sugar moiety.

### Embodiment 268

The compound of any of claim 1-107, 140-146, 180-205, or 241-267, wherein the 14^{th} nucleoside from the 5′-end of the target recognition portion comprises a modified sugar moiety.

### Embodiment 269

The compound of any of claim 1-107, 140-146, 180-205, or 241-268, wherein the 15^{th} nucleoside from the 5′-end of the target recognition portion comprises a modified sugar moiety.

### Embodiment 270

The compound of any of claim 1-107, 140-146, 180-205, or 241-269, wherein the 16^{th} nucleoside from the 5′-end of the target recognition portion comprises a modified sugar moiety.

### Embodiment 271

The compound of any of claims 268-270, wherein each modified sugar moiety at position 14, 15, and/or 16 from the 5′-end of the target recognition portion is a linearly modified sugar moiety.

### Embodiment 272

The compound of claim 271, wherein each modified sugar moiety at position 14, 15, and/or 16 from the 5′-end of the target recognition portion is independently selected from 2′-H(H) and 2′-F modified sugar moieties.

### Embodiment 273

The compound of claim 272, wherein each modified sugar moiety at position 14, 15, and/or 16 from the 5′-end of the target recognition portion is a 2′-H(H) modified sugar moiety.

### Embodiment 274

The compound of any of claim 1-107, 140-146, 180-205, or 241-273, wherein the modified crRNA comprises at least three of the following features:
(a) two linker nucleosides linked to the 5′-end of the CRISPR recognition portion of the modified crRNA;
(b) 1^{st}, 8^{th}, and/or 9^{th} nucleoside from the 5′-end of the target recognition portion of the modified crRNA independently comprising 2′-F or 2′-H(H) modified sugar moiety;
(c) at least one terminal phosphorothioate internucleoside linkage at each of the 3′ and 5′ termini of the modified crRNA
(d) at least one nucleoside at position 5, 6, 7, 8, 11, or 12 from the 3′-end of the CRISPR recognition portion comprises a modified sugar moiety
(e) one to five 3′-terminal nucleosides of the modified crRNA comprising independently selected modified sugar moieities

### Embodiment 275

The compound of claim 274, wherein the modified crRNA comprises features (a),
(c), and (e).

### Embodiment 276

The compound of claim 274, wherein the modified crRNA comprises features (a),
(c), and (d).

### Embodiment 277

The compound of claim 274, wherein the modified crRNA comprises features (a),
(b), and (c).

### Embodiment 278

The compound of claim 274, wherein the modified crRNA comprises features (a),
(b), (c), and (e).

### Embodiment 279

The compound of claim 274, wherein the modified crRNA comprises features (a),
(c), (d), and (e).

### Embodiment 280

The compound of claim 274, wherein the modified crRNA comprises features (a),
(b), (c), (d), and (e).

### Embodiment 281

A pharmaceutical composition comprising the compound of any of claims 241-280.

### Embodiment 282

The pharmaceutical composition of claim 281, wherein the pharmaceutical composition comprises a ribonucleoprotein complex.

### Embodiment 283

The pharmaceutical composition of claim 282, wherein the ribonucleoprotein complex comprises a Cpf1 nuclease and the compound comprising the modified crRNA.

### Embodiment 284

A method comprising contacting a cell with the compound or composition of any of claims 241-283.

### Embodiment 285

A method comprising contacting a cell with the compound or composition of any of claims 241-283, wherein the cell expresses a Cpf1 nuclease.

### Embodiment 286

A method comprising contacting a cell with the compound or composition of any of claims 241-283 and a plasmid that encodes a Cpf1 nuclease.

### Embodiment 287

A method comprising contacting a cell with the compound or composition of any of claims 241-283 and an mRNA that encodes a Cpf1 nuclease.

### Embodiment 288

The method of any of claims 284-287, wherein the modified crRNA is taken up by the cell in the absence of a transfection reagent.

### Embodiment 289

The method of any of claims 284-288, wherein the cell is in an animal.

### Embodiment 290

A method comprising administering to an animal the compound or composition of any of claims 241-283.

### Embodiment 291

The method of claim 290, wherein the administration is subcutaneous.

### Embodiment 292

The method of claim 290, wherein the administration is intrathecal.

### Embodiment 293

The method of any of claims 290-292 comprising administering a plasmid that encodes a Cpf1 nuclease.

### Embodiment 294

The method of any of claims 290-292 wherein the animal expresses a Cpf1 nuclease.

### Embodiment 295

The method of claim 286 or 293, wherein the plasmid is delivered to cells within the animal via an adeno-associated virus (AAV).

### Embodiment 296

The method of claim 286 or 293, wherein the plasmid is delivered to cells within the animal via a lentivirus.

### Embodiment 297

The method of any of claims 284-296, wherein a target gene is edited.

### Embodiment 298

The method of claim 297, wherein the modified crRNA is degraded in a cell after the target gene is edited in the cell.

### Embodiment 299

The method of any of claim 285-287 or 293-298, wherein the Cpf1 nuclease does not exhibit nuclease activity in the absence of the modified crRNA.

### Embodiment 300

The method of any of claims 284-299 comprising contacting the cell with a second compound that degrades or inhibits the activity or expression of the modified crRNA or a Cpf1 nuclease.

### Embodiment 301

The method of claim 300, wherein the cell is contacted with the second compound after a target gene has been edited.

### Embodiment 302

The method of claim 300 or 301, wherein the second compound comprises an oligonucleotide that is complementary to the modified crRNA.

### Embodiment 303

The method of claim 300 or 301, wherein the second compound comprises a crRNA that targets a Cpf1 nuclease gene.

### Embodiment 304

The method of claim 300 or 301, wherein the second compound comprises an oligonucleotide that is complementary to a Cpf1 transcript.

### Embodiment 305

The method of claim 303 or 304, wherein the expression of the Cpf1 nuclease is inhibited.

### Embodiment 306

The method of any of claims 289-305, wherein the animal is a human.

### Embodiment 307

The method of any of claims 284-306, wherein editing of at least one off-target gene is reduced relative to editing the at least one off-target gene when unmodified crRNA or a compound comprising more than 45 nucleosides is used in place of the modified crRNA.

### Embodiment 308

The method of any of claim 290 or 293-297, wherein the administration is intravitreal.

### Embodiment 309

The method of any of claims 284-288, wherein the cell is a plant cell.

### Embodiment 310

The method of any of claims 284-289, wherein the cell is a T-cell.

### Embodiment 311

A method of treating a disease in an individual comprising administering the compound of any of claims 241-280 or the composition of any of claims 281-283 to the individual.

### Embodiment 312

A method of treating a disease in an individual comprising administering the compound of any of claims 241-280 or the composition any of claims 281-283 to the individual, thereby treating the disease in the individual.

### Embodiment 313

Use of the compound of any of claims 241-280 or the composition of any of claims 281-283 for the treatment of a disease.

### Embodiment 314

Use of the compound of any of claims 241-280 or the composition of any of claims 281-283 for preparation of a medicament.

### Embodiment 315

A method of administering the compound of any of claims 241-280 or the composition of any of claims 281-283 to an animal, and harvesting an organ from the animal for transplantation into a human.

### Embodiment 316

The pharmaceutical composition of any of embodiments 108, 147, 206, or 281 comprising a liposome or lipid nanoparticle.

### Embodiment 317

The pharmaceutical composition of any of embodiments 108, 147, 206, 281, or 316 comprising mRNA that encodes a Cpf1 nuclease.

### Embodiment 318

The pharmaceutical composition of embodiment 317, wherein the compound comprising the modified crRNA and the mRNA encoding a Cpf1 nuclease are contained with a liposome or lipid nanoparticle.

### Embodiment 319

The method of any of embodiments 212-214, 151-153, 212-214, or 287-289, wherein the mRNA encoding the Cpf1 nuclease and the compound comprising the modified crRNA are contained within a liposome or lipid nanoparticle.

### Embodiment 320

A method of treating a disease in an individual comprising administering the pharmaceutical composition of any of embodiments 316-318 to the individual.

### Embodiment 321

A method of treating a disease in an individual comprising administering the pharmaceutical composition of any of embodiments 316-318 to the individual, thereby treating the disease in the individual.

A. Certain Modified Nucleosides

Certain compounds of the present invention incorporate modified nucleosides. Unless otherwise provided, the following modified nucleosides, without limitation, are suitable for such incorporation into modified oligonucleotides for use as crRNA. In certain embodiments, modified oligonucleotides comprise at least one modified nucleoside. Such modified nucleosides comprise a modified sugar moiety or a modified nucleobase or both a modified sugar moiety and a modified nucleobase.

1. Certain Sugar Moieties

In certain embodiments, modified oligonucleotides, such as modified crRNAs, comprise one or more modified nucleosides comprising a modified sugar moiety. Such modified oligonucleotides comprising one or more sugar-modified nucleosides may have desirable properties, such as enhanced nuclease stability or increased binding affinity with a target nucleic acid relative to oligonucleotides lacking such sugar-modified nucleosides. In certain embodiments, modified sugar moieties are linearly modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of substituted sugar moieties.

In certain embodiments, modified sugar moieties are linearly modified sugar moieties comprising a furanosyl ring with one or more acyclic substituent, including but not limited to substituents at the 2′ and/or 5′ positions. Examples of 2′-substituent groups suitable for linearly modified sugar moieties for use in modified crRNA include but are not limited to: 2′-H, 2′-F, 2′-OCH₃ (“OMe” or “O-methyl”), and 2′-O(CH₂)₂OCH₃ (“MOE”). The 2′-substituent groups of such linearly modified sugar moieties replace the 2′-OH group that is present in unmodified sugar moities. In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 5′-substituent groups suitable for linearly modified sugar moieties include but are not limited to: 5′-methyl (R or S), 5′-vinyl, and 5′-methoxy. In certain embodiments, linearly modified sugars comprise more than one non-bridging sugar substituent, for example, 2′-F-5′-methyl sugar moieties (see, e.g., PCT International Application WO 2008/101157, for additional 2′, 5′-bis substituted sugar moieties and nucleosides).

In certain embodiments, a 2′-substituted nucleoside or 2′-linearly modified nucleoside comprises a sugar moiety comprising a linear 2′-substituent group selected from: H, F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-linearly modified nucleoside comprises a sugar moiety comprising a linear 2′-substituent group selected from: H, F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-linearly modified nucleoside comprises a sugar moiety comprising a linear 2′-substituent group selected from: H, F, OCH₃, and OCH₂CH₂OCH₃.

Nucleosides comprising modified sugar moieties, such as linearly modified sugar moieties, are referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modifed sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., U.S. Pat. No. 7,399,845), 4′-C(CH₃)(CH₃)—O-2′ and analogs thereof (see, e.g., WO2009/006478), 4′-CH₂—N(OCH₃)-2′ and analogs thereof (see, e.g., WO2008/150729), 4′-CH₂—O—N(CH₃)-2′ (see, e.g., US2004/0171570), 4′-CH₂—C(H)(CH₃)-2′ (see, e.g., Chattopadhyaya, et al., J. Org. Chem., 2009, 74, 118-134), 4′-CH₂—C(═CH₂)-2′ and analogs thereof (see, published PCT International Application WO 2008/154401), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US2004/0171570, US2007/0287831, and US2008/0039618; U.S. patent Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In certain embodiments, bicyclic sugar moieties and nucleosides incorporating such bicyclic sugar moieties are further defined by isomeric configuration. For example, an LNA nucleoside (described above) may be in the α-L configuration or in the β-D configuration.

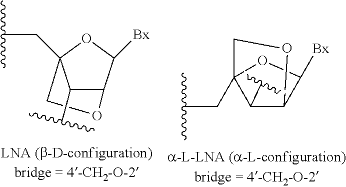

α-L-methyleneoxy (4′-CH₂—O-2′) or α-L-LNA bicyclic nucleosides have been incorporated into oligonucleotides (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars). (see, e.g., WO 2007/134181, wherein LNA nucleosides are further substituted with, for example, a 5′-methyl or a 5′-vinyl group, and see, e.g., U.S. Pat. Nos. 7,547,684; 7,750,131; 8,030,467; 8,268,980; 7,666, 854; and 8,088,746).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described above. For example, certain sugar surrogates comprise a 4′-sulfur atom and a substitution at the 2′-position (see, e.g., US2005/0130923) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see Leumann, C J. Bioorg. & Med. Chem. 2002, 10, 841-854), fluoro HNA:

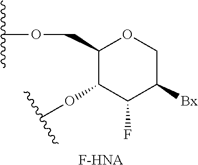

(“F-HNA”, see e.g., U.S. Pat. Nos. 8,088,904; 8,440,803; and 8,796,437, F-HNA can also be referred to as a F-THP or 3′-fluoro tetrahydropyran), and nucleosides comprising additional modified THP compounds having the formula:

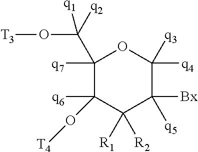

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and U.S. Pat. Nos. 5,698,685; 5,166,315; 5,185,444; and 5,034,506). As used here, the term “morpholino” means a sugar surrogate having the following structure:

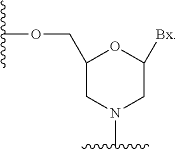

In certain embodiments, morpholinos may be modified, for example by adding or altering various substituent groups from the above morpholino structure. Such sugar surrogates are referred to herein as “modifed morpholinos.”

In certain embodiments, sugar surrogates comprise acyclic moieites. Examples of nucleosides and oligonucleotieds comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in WO2011/133876.

Many other bicyclic and tricyclic sugar and sugar surrogate ring systems are known in the art that can be used in modified nucleosides (see, e.g., Leumann, J. C, Bioorganic & Medicinal Chemistry, 2002, 10, 841-854).

2. Certain Modified Nucleobases

In certain embodiments, modified oligonucleotides, such as modified crRNAs, comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Representative United States patents that teach the preparation of certain of the above noted modified nucleobases as well as other modified nucleobases include without limitation, US2003/0158403, U.S. Pat. Nos. 3,687,808; 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096.

B. Certain Modified Internucleoside Linkages

In certain embodiments, nucleosides of modified oligonucleotides, such as modified crRNAs, may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester (—O—C(═O)—S—), thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

C. Certain Conjugate Groups and Terminal Groups

In certain embodiments, oligonucleotides for use as crRNA further comprise conjugate groups and/or terminal groups. In certain embodiments, compounds comprising oligonucleotides for use as crRNA further comprise a conjugate group or terminal group. In certain such embodiments, oligonucleotides are covalently attached to one or more conjugate group. In certain embodiments, conjugate groups modify one or more properties of the attached oligonucleotide, including but not limited to pharmacodynamics, pharmacokinetics, stability, binding, absorption, cellular distribution, cellular uptake, charge and clearance. In certain embodiments, conjugate groups impart a new property on the attached oligonucleotide, e.g., fluorophores or reporter groups that enable detection of the oligonucleotide. Conjugate groups and/or terminal groups may be added to oligonucleotides having any of the modifications or motifs described above.

Conjugate groups include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; doi: 10.1038/mtna.2014.72 and Nishina et al., Molecular Therapy, 2008, 16, 734-740), or a GalNAc cluster (e.g., WO2014/179620).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

Conjugate groups are attached directly or via an optional conjugate linker to a parent compound, such as a crRNA oligonucleotide. In certain embodiments, conjugate groups are directly attached to oligonucleotides. In certain embodiments, conjugate groups are indirectly attached to oligonucleotides via conjugate linkers. In certain embodiments, the conjugate linker comprises a chain structure, such as a hydrocarbyl chain, or an oligomer of repeating units such as ethylene glycol or amino acid units. In certain embodiments, conjugate groups comprise a cleavable moiety. In certain embodiments, conjugate groups are attached to oligonucleotides via a cleavable moiety. In certain embodiments, conjugate linkers comprise a cleavable moiety. In certain such embodiments, conjugate linkers are attached to oligonucleotides via a cleavable moiety.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the crRNA oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, a cleavable moiety is a cleavable bond. In certain embodiments, a cleavable moiety comprises a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate linker or conjugate group.

Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

In certain embodiments, a conjugate group is a cell-targeting moiety. In certain embodiments, a conjugate group, optional conjugate linker, and optional cleavable moiety have the general formula:

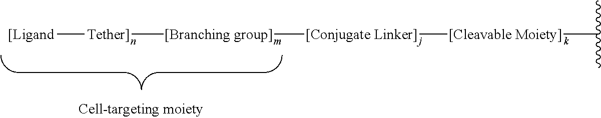

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate. In certain embodiments, each ligand is, independently selected from galactose, N-acetyl galactoseamine (GalNAc), mannose, glucose, glucoseamine and fucose. In certain embodiments, each ligand is N-acetyl galactoseamine (GalNAc). In certain embodiments, the cell-targeting moiety comprises 3 GalNAc ligands. In certain embodiments, the cell-targeting moiety comprises 2 GalNAc ligands. In certain embodiments, the cell-targeting moiety comprises 1 GalNAc ligand.

Certain Pharmaceutical Compositions

In certain embodiments, the present invention provides pharmaceutical compositions comprising one or more crRNA. In certain embodiments, such pharmaceutical composition comprises a tracrRNA. In certain embodiments, the pharmaceutical composition comprises a means of expressing a CRISPR nuclease. In certain embodiments, such means of expressing the CRISPR nuclease is a plasmid or a viral vector. In certain such embodiments, the pharmaceutical composition comprises a suitable pharmaceutically acceptable diluent or carrier. In certain embodiments, a pharmaceutical composition comprises a modified crRNA. In certain such embodiments, the modified crRNA is a component of a ribonucleoprotein particle or or complex (RNP). In certain such embodiments, the RNP also comprises a nuclease. In certain such embodiments, the nuclease is a Cpf1 nuclease. In certain embodiments, a pharmaceutical composition comprises a liposome or lipid nanoparticle. In certain such embodiments, the liposome or lipid nanoparticle contains the modified crRNA. In certain such embodiments, the liposome or lipid nanoparticle contains an mRNA encoding a Cpf1 nuclease. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one antisense compound and sterile water. In certain embodiments, the sterile water is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile PBS. In certain embodiments, the sterile PBS is pharmaceutical grade PBS.

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified or naturally occurring bases, such as “AT^{m}CGAUCG,” wherein ^{m}C indicates a cytosine base comprising a methyl group at the 5-position.

### EXAMPLES

The following examples illustrate certain embodiments of the present invention and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Gene Editing Effects of Truncated crRNAs

Truncated crRNAs comprising a target recognition portion that is complementary to DNA (cytosine-5)-methyltransferase 1 (DNMT1) were designed and synthesized to test their effects on gene editing of DNMT1. HEK293T cells were transfected with a plasmid encoding Cpf1 and a double-stranded gblock (IDT, Coralville, Iowa) encoding a crRNA listed in the table below. 48 hours later, genomic DNA was isolated from cells and used in a SURVEYOR assay (Integrated DNA Technologies) according to the manufacturer's directions. The PCR primers used to amplify the crRNA target site in the DNMT1 gene were forward: 5′-CTGGGACTCAGGCGGGTCAC-3′ (SEQ ID NO: 1) and reverse: 5′-CCTCACACAACAGCTTCATGTCAGC-3′ (SEQ ID NO: 2). Following Cell cleavage, the DNA was run on a gel. Gene editing of DNMT1 was evaluated by measuring the extent of non-homologous end joining (NHEJ) within DMT1. Quantification of the bands in the gel was performed using Image J software, and the NHEJ incidence percentage was calculated using the following formula: NHEJ (%)=100×(1-(fraction cut of target gene)^{0.5}), wherein the fraction cut of the target gene was determined by dividing the fluorescent signal of the cut target gene fragment(s) by the total fluorescent signal of the cut and intact target gene fragment(s). The NHEJ incidence for each truncated crRNA was normalized to the NHEJ incidence of the positive control, full-length crRNA 002, and the normalized value was referred to as the gene disruption percentage. The results, shown in the table below, indicate that multiple truncated crRNAs edited the target gene. An entry of “n.d.” indicates no data due to the lack of detectable cleavage bands in the gel.

TABLE 1
crRNAs targeting DNMT1

| | | | | Normalized | |
|---|---|---|---|---|---|
| | | | | gene | SEQ |
| | | | | disruption | ID |
| Name | | Sequence (5′ to 3′) | Length | (%) | NO. |
| 002 | 1090808 | UAAUUUCUACUCUUGUAGAUCUGAUGGUCCA | 43 | 100 | 18 |
| | | UGUCUGUUACUC | | | |
| 005 | 1091140 | UUCUACUCUUGUAGAUCUGAUGGUCCAUGUC | 39 | n.d. | 19 |
| | | UGUUACUC | | | |
| 006 | 1091141 | UAAUUUCUACUCUUGUAGAUCUGAUGGUCCA | 40 | 102 | 20 |
| | | UGUCUGUUA | | | |
| 007 | 1091142 | UUCUACUCUUGUAGAUCUGAUGGUCCAUGUC | 34 | n.d. | 21 |
| | | UGU | | | |
| 008 | 1090812 | UAAUUUCUACUCUUGUAGAUCUGAUGGUCCA | 38 | 90 | 22 |
| | | UGUCUGU | | | |
| 009 | 1091143 | UUCUACUCUUGUAGAUCUGAUGGUCCAUGUC | 36 | n.d. | 23 |
| | | UGUUA | | | |
| 010 | 1090813 | AAUUUCUACUCUUGUAGAUCUGAUGGUCCAU | 37 | 84 | 24 |
| | | GUCUGU | | | |
| 1034621 | 1034621 | AUUUCUACUCUUGUAGAUCUGAUGGUCCAUG | 36 | 60 | 25 |
| | | UCUGU | | | |
| 012 | 1090814 | UUUCUACUCUUGUAGAUCUGAUGGUCCAUGU | 35 | 17 | 26 |
| | | CUGU | | | |
| 013 | 1090809 | AAUUUCUACUCUUGUAGAUCUGAUGGUCCAU | 42 | 99 | 27 |
| | | GUCUGUUACUC | | | |
| 014 | 1090810 | AUUUCUACUCUUGUAGAUCUGAUGGUCCAUG | 41 | 65 | 28 |
| | | UCUGUUACUC | | | |
| 015 | 1090811 | UUUCUACUCUUGUAGAUCUGAUGGUCCAUGU | 40 | 20 | 29 |
| | | CUGUUACUC | | | |

All of the nucleosides in the table above are unmodified ribonucleosides comprising 2-hydroxy sugar moieties, and all of the internucleoside linkages in the table above are phosphate internucleoside linkages. The underlined portion of each crRNA is the target recognition portion, and the portion that is not underlined is the CRISPR recognition portion of each crRNA. In Table 1, the CRISPR recognition portions of the crRNAs recognize Cpf1.

### Example 2: Gene Editing Effects of Modified crRNAs

Modified crRNAs comprising a target recognition portion that is complementary to DNMT1 were designed and synthesized to test their effects on gene editing of DNMT1. HEK293T cells were transfected with a plasmid encoding Cpf1 using Lipofectamine 3000 (Life Technologies, Carlsbad, Calif.). The next morning, the cells were transfected with a modified crRNA listed in the table below using Lipofectamine RNAi max (Life Technologies). 24 hours later, genomic DNA was isolated from cells and analyzed as described in Example 1 in order to determine the extent of gene editing of DNMT1. The NHEJ incidence for each modified crRNA was normalized to the NHEJ incidence observed for crRNA 1034621, which was also tested in Example 1. The normalized values are referred to as the gene disruption percentages. The results, shown in the table below, indicate that multiple modified crRNAs edited the target gene. An entry of “n.d.” indicates no data due to the lack of detectable cleavage bands in the gel.

TABLE 2
crRNAs targeting DNMT1

| | | | (Normalized) | SEQ |
|---|---|---|---|---|
| | | | gene | ID |
| Name | Sequence (5′ to 3′) | Length | disruption (%) | NO. |
| 1034621 | AᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒAᵣₒ | 36 | 100 | 25 |
| | UᵣₒGᵣₒGᵣₒUᵣₒCᵣₒCᵣₒAᵣₒUᵣₒGᵣₒUᵣₒCᵣₒUᵣₒGᵣₒUᵣ | | | |
| 1038257 | AᵣₛUᵣₛUᵣₛUᵣₛCᵣₛUᵣₛAᵣₛCᵣₛUᵣₛCᵣₛUᵣₛUᵣₛGᵣₛUᵣₛAᵣₛGᵣₛAᵣₛUᵣₛCᵣₛUᵣₛGᵣₛAᵣₛUᵣₛ | 36 | n..d. | 25 |
| | GᵣₛGᵣₛUᵣₛCᵣₛCᵣₛAᵣₛUᵣₛGᵣₛUᵣₛCᵣₛUᵣₛGᵣₛUᵣ | | | |
| 1038259 | AₘₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒAᵣₒ | 36 | n.d. | 25 |
| | UᵣₒGᵣₒGᵣₒUᵣₒCᵣₒCᵣₒAᵣₒUᵣₒGᵣₒUᵣₒCᵣₒUᵣₒGᵣₒUᵣ | | | |
| 1038260 | AᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒAᵣₒ | 36 | 108 | 25 |
| | UᵣₒGᵣₒGᵣₒUᵣₒCᵣₒCᵣₒAᵣₒUᵣₒGᵣₒUᵣₒCᵣₒUᵣₒGᵣₒUₘ | | | |
| 1038261 | AₘₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒAᵣₒ | 36 | n.d. | 25 |
| | UᵣₒGᵣₒGᵣₒUᵣₒCᵣₒCᵣₒAᵣₒUᵣₒGᵣₒUᵣₒCᵣₒUᵣₒGᵣₒUₘ | | | |

A subscript “m” indicates a 2′-O-methyl modification. A subscript “r” indicates an unmodified, 2′-hydroxy sugar moiety. A subscript “o” indicates a phosphate internucleoside linkage, and a subscript “s” indicates a phosphorothioate internucleoside linkage. The underlined portion of each crRNA is the target recognition portion, and the portion that is not underlined is the CRISPR recognition portion of the crRNA. In the table above, the CRISPR recognition portions of the crRNAs recognize Cpf1.

### Example 3: Gene Editing Effects of Modified crRNAs

Modified crRNAs comprising a target recognition portion that is complementary to DNMT1 were designed and synthesized to test their effects on gene editing of DNMT1. HEK293T cells were transfected as described in Example 2, except the modified crRNAs are listed in the table below. Genomic DNA was isolated and analyzed as described in Example 1. The NHEJ incidence for each modified crRNA was normalized to the NHEJ incidence observed for crRNA 1034621, which was also tested in Examples 1 and 2. The normalized values are referred to as the gene disruption percentages. The results, shown in the table below, indicate that modified crRNAs edited the target gene. Nearly all of the modified crRNAs in the table below edited the target gene with greater efficacy than the unmodified control (crRNA 1034621).

TABLE 3
crRNAs targeting DNMT1

| | | | (Normalized) | SEQ |
|---|---|---|---|---|
| | | | gene | ID |
| Name | Sequence (5′ to 3′) | Length | disruption (%) | NO. |
| 1034621 | AᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒAᵣₒ | 36 | 100 | 25 |
| | UᵣₒGᵣₒGᵣₒUᵣₒCᵣₒCᵣₒAᵣₒUᵣₒGᵣₒUᵣₒCᵣₒUᵣₒGᵣₒUᵣ | | | |
| 1038268 | AᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒAᵣₒ | 36 | 141 | 25 |
| | UᵣₒGᵣₒGᵣₒUᵣₒCᵣₒCᵣₒAᵣₒUᵣₒGᵣₒUᵣₒCᵣₒUᵣₒGᵣₛUₘ | | | |
| 1038269 | AᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒAᵣₒ | 36 | 148 | 25 |
| | UᵣₒGᵣₒGᵣₛUᵣₒCᵣₛCᵣₒAᵣₛUᵣₒGᵣₛUᵣₒCisUᵣₒGᵣₛUₘ | | | |
| 1038270 | AᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒAᵣₒ | 36 | 109 | 25 |
| | UᵣₒGᵣₒGᵣₒUᵣₒCᵣₒCᵣₒAᵣₒUᵣₒGᵣₒUᵣₒCᵣₒUᵣₒGₘₒUₘ | | | |
| 1038271 | AᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒAᵣₒ | 36 | 141 | 25 |
| | UᵣₒGᵣₒGᵣₒUᵣₒCᵣₒCᵣₒAᵣₒUᵣₒGᵣₒUᵣₒCᵣₒUₘₒGₘₒUₘ | | | |
| 1038272 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒ | 38 | 152 | 22 |
| | GᵣₒAᵣₒUᵣₒGᵣₒGᵣₒUᵣₒCᵣₒCᵣₒAᵣₒUᵣₒGᵣₒUᵣₒCᵣₒUᵣₒGᵣₒUₘ | | | |
| 1038273 | UₘₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒ | 38 | 88 | 22 |
| | GᵣₒAᵣₒUᵣₒGᵣₒGᵣₒUᵣₒCᵣₒCᵣₒAᵣₒUᵣₒGᵣₒUᵣₒCᵣₒUᵣₒGᵣₒUₘ | | | |
| 1038274 | UₘₛAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒ | 38 | 109 | 22 |
| | GᵣₒAᵣₒUᵣₒGᵣₒGᵣₒUᵣₒCᵣₒCᵣₒAᵣₒUᵣₒGᵣₒUᵣₒCᵣₒUᵣₒGᵣₒUₘ | | | |
| 1038275 | CᵣₒUᵣₒUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 169 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₒGᵣₒUₘ | | | |
| 1038276 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 144 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₒGᵣₒUₘ | | | |
| 990509 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 165 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGᵣₛUₘ | | | |

A subscript “m” indicates a 2′-O-methyl modification. A subscript “r” indicates an unmodified, 2′-hydroxy sugar moiety. A subscript “o” indicates a phosphate internucleoside linkage, and a subscript “s” indicates a phosphorothioate internucleoside linkage. The underlined portion of each crRNA is the target recognition portion, and the bolded nucleosides are linker nucleosides. The portion that is neither bold nor underlined is the CRISPR recognition portion of each crRNA. In the table above, the CRISPR recognition portions of the crRNAs recognize Cpf1. Modified crRNAs 1038273, 1038274, 1038276 and 990509 are 5′-stabilized. The CRISPR recognition portions of crRNAs 1038273 and 1038274 comprise one or more 5′-stabilizing modifications. The linker nucleosides of crRNAs 1038276 and 990509 comprise one or more 5′-stabilizing modifications.

### Example 4: Gene Editing Effects of Modified crRNAs

Modified crRNAs comprising a target recognition portion that is complementary to DNMT1 were designed and synthesized to test their effects on gene editing of DNMT1. HEK293T cells were transfected as described in Example 2, except the modified crRNAs are listed in the table below. Genomic DNA was isolated and analyzed as described in Example 1. The NHEJ incidence for each modified crRNA was normalized to the NHEJ incidence observed for crRNA 1038299, which had the highest activity of those tested in this experiment. The normalized values are referred to as the gene disruption percentages. The

TABLE 4
crRNAs targeting DNMT1

| | | | (Normalized) | SEQ |
|---|---|---|---|---|
| | | | gene | ID |
| Name | Sequence (5′ to 3′) | Length | disruption (%) | NO. |
| 1038292 | CₘₛUₘₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 39 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | | |
| 1038293 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | n.d. | 30 |
| | CᵣₛUᵣₛGᵣₛAᵣₛUᵣₛGsGᵣₛUᵣₛCᵣₛCᵣₛAᵣₛUᵣₛGᵣₛUᵣₛCᵣₛUᵣₛGₘₛUₘ | | | |
| 1038294 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | n.d. | 30 |
| | CᵣₒUᵣₒGᵣₒAᵣₒUᵣₒGᵣₛGᵣₛUᵣₛCᵣₛCᵣₛAᵣₛUᵣₛGᵣₛUᵣₛCᵣₛUᵣₛGₘₛUₘ | | | |
| 1038295 | CₘₛUₘₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | n.d. | 30 |
| | CᵣₒUᵣₒGᵣₒAᵣₒUᵣₒGᵣₛGᵣₛUᵣₛCᵣₛCᵣₛAᵣₛUᵣₛGᵣₛUᵣₛCᵣₛUᵣₛGₘₛUₘ | | | |
| 1038296 | CₘₛUₘₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | n.d. | 30 |
| | CᵣₛUᵣₛGᵣₛAᵣₛUᵣₛGᵣₛGᵣₛUᵣₛCᵣₛCᵣₛAᵣₛUᵣₛGᵣₛUᵣₛCᵣₛUᵣₛGₘₛUₘ | | | |
| 1038297 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 80 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGᵣₛU_{f} | | | |
| 1038298 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 86 | 30 |
| | CᵣₒUᵣₒGᵣₒAᵣₒUᵣₒGᵣₒGᵣₒUᵣₒCᵣₒCᵣₒAᵣₒUᵣₒGᵣₒU_{fo}C_{fo}U_{fo}G_{fo}U_{f} | | | |
| 1038299 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 100 | 30 |
| | CᵣₒUᵣₒGᵣₒAᵣₒUᵣₒGᵣₒGᵣₛUᵣₒCᵣₛCᵣₒAᵣₛUᵣₒGᵣₛU_{fo}C_{fs}U_{fo}G_{fs}U_{f} | | | |
| 1038300 | CₘₛUᵣₛUᵣₒAᵣₒAᵣₒUᵣₒU_{fo}UᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 49 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | | |
| 1038301 | CₘₛUᵣₛUᵣₒAᵣₒAᵣₒUᵣₒUᵣₒU_{fo}CᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 44 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | | |
| 1038302 | CₘₛUᵣₛUᵣₒAᵣₒAᵣₒUᵣₒUᵣₒUᵣₒC_{fo}UᵣₒAᵣₒCᵣₒUᵣₒCᵣₒUᵣₒUᵣₒGᵣₒUᵣₒAᵣₒGᵣₒAᵣₒUᵣₒ | 40 | 58 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | | |
| 1038303 | CₘₛUᵣₛUᵣₒAᵣₒAᵣₒUᵣₒUᵣₒUᵣₒCᵣₒUᵣₒAᵣₒC_{fo}UᵣₒCᵣₒUᵣₒUᵣₒGᵣₒUᵣₒAᵣₒGᵣₒAᵣₒUᵣₒ | 40 | 52 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | | |
| 1038304 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒU_{fo}GᵣₒUᵣₒAᵣₒGᵣₒAᵣₒUᵣₒ | 40 | 37 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | | |
| 1038305 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒU_{fo} | 40 | n.d. | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | | |

A subscript “table” indicates a 2′-O-methyl modification. A subscript “r” indicates an unmodified, 2′-hydroxy sugar moiety. A subscript “f” indicates a 2′-F modification. A subscript “o” indicates a phosphate internucleoside linkage, and a subscript “s” indicates a phosphorothioate internucleoside linkage. The underlined portion of each crRNA is the target recognition portion, and the bolded nucleosides are linker nucleosides. The portion that is neither bold nor underlined is the CRISPR recognition portion of each crRNA. In the table above, the CRISPR recognition portions of the crRNAs recognize Cpf1. The modified crRNAs in the table above are 5′-stabilized, and the linker nucleosides comprise the 5′-stabilizing modifications.

### Example 5: Gene Editing Effects of Modified crRNAs

Modified crRNAs comprising a target recognition portion that is complementary to DNMT1 were designed and synthesized to test their effects on gene editing of DNMT1. HEK293T cells were transfected as described in Example 2, except the modified crRNAs are listed in the table below. Genomic DNA was isolated and analyzed as described in Example 1. The NHEJ incidence for each modified crRNA was normalized to the NHEJ incidence observed for crRNA 1034621, which was also tested in Examples 1-3. The normalized values are referred to as the gene disruption percentages. An entry of “n.d.” indicates no data due to the lack of detectable cleavage bands in the gel. The results, shown in the table below, indicate that multiple modified crRNAs edited the target gene, and, in many cases, were more efficacious than the unmodified crRNA 1034621. The results also indicated that certain positions that tolerate nucleobase changes in the target recognition portion (see, e.g., Kleinstiver et al. in Nature Biotechnology, 34, 869 (2016)), also tolerate modified sugars.

TABLE 5
crRNAs targeting DNMT1

| | | | (Normalized) | SEQ |
|---|---|---|---|---|
| | | | gene | ID |
| Name | Sequence (5′ to 3′) | Length | disruption (%) | NO. |
| 1034621 | AᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒAᵣₒ | 36 | 100 | 25 |
| | UᵣₒGᵣₒGᵣₒUᵣₒCᵣₒCᵣₒAᵣₒUᵣₒGᵣₒUᵣₒCᵣₒUᵣₒGᵣₒUᵣ | | | |
| 991458 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 77 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | | |
| 991461 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 124 | 31 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGᵣₛTₖ | | | |
| 991462 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 168 | 31 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛTₖ | | | |
| 991775 | ^{m}CₖₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 147 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGᵣₛUₘ | | | |
| 991776 | ^{m}CₖₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 159 | 31 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₖₛTₖ | | | |
| 991777 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 138 | 31 |
| | CᵣₒUᵣₒGᵣₒAᵣₒUᵣₒGᵣₒG_{fo}U_{fo}C_{fo}C_{fo}A_{fo}U_{fo}G_{fo}U_{fo}C_{fo}U_{fs}GₖₛTₖ | | | |
| 991783 | ^{m}CₖₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 176 | 30 |
| | CᵣₒUᵣₒGᵣₒAᵣₒUᵣₒGᵣₒGᵣₒUᵣₒ^{m}CₖₒCᵣₒAᵣₒUᵣₒGᵣₒUᵣₒCᵣₒUᵣₛGₘₛUₘ | | | |
| 991784 | ^{m}CₖₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | n.d. | 32 |
| | ^{m}CₖₒUᵣₒGᵣₒAᵣₒUᵣₒGᵣₒGᵣₒTₖₒ^{m}CₖₒCᵣₒAᵣₒUᵣₒGᵣₒUᵣₒCᵣₒUᵣₛGₘₛUₘ | | | |

A subscript “m” indicates a 2′-O-methyl modification. A superscript “m” adjacent to a “C” indicates a 5-methyl cytosine. A subscript “r” indicates an unmodified, 2′-hydroxy sugar moiety. A subscript “f” indicates a 2′-F modification. A subscript “k” indicates a cEt modification. A subscript “o” indicates a phosphate internucleoside linkage, and a subscript “s” indicates a phosphorothioate internucleoside linkage. The underlined portion of each crRNA is the target recognition portion, and the bolded nucleosides are linker nucleosides. The portion that is neither bold nor underlined is the CRISPR recognition portion of each crRNA. In the table above, the CRISPR recognition portions of the crRNAs recognize Cpf1. Other than crRNA 1034621, the modified crRNAs in the table above are 5′-stabilized, and the linker nucleosides comprise the 5′-stabilizing modifications.

### Example 6: Gene Editing Effects of Modified crRNAs

Modified crRNAs comprising a target recognition portion that is complementary to DNMT1 were designed and synthesized to test their effects on gene editing of DNMT1. HEK293T cells were transfected as described in Example 2, except the modified crRNAs are listed in the table below. Genomic DNA was isolated and analyzed as described in Example 1. The NHEJ incidence for each modified crRNA was normalized to the NHEJ incidence observed for crRNA 989549. The normalized values are referred to as the gene disruption percentages. The results, shown in the table below, indicate that modified crRNAs edited the target gene.

TABLE 6
crRNAs targeting DNMT1

| | | | (Normalized) | SEQ |
|---|---|---|---|---|
| | | | gene | ID |
| Name | Sequence (5′ to 3′) | Length | disruption (%) | NO. |
| 989549 | CᵣₒUᵣₒUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 100 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₒGᵣₒUᵣ | | | |
| 1038293 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 26 | 30 |
| | CᵣₛUᵣₛGᵣₛAᵣₛUᵣₛGᵣₛGᵣₛUᵣₛCᵣₛCᵣₛAᵣₛUᵣₛGᵣₛUᵣₛCᵣₛUᵣₛGₘₛUₘ | | | |
| 1038294 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 38 | 30 |
| | CᵣₒUᵣₒGᵣₒAᵣₒUᵣₒGᵣₛGᵣₛUᵣₛCᵣₛCᵣₛAᵣₛUᵣₛGᵣₛUᵣₛCᵣₛUᵣₛGₘₛUₘ | | | |
| 1086669 | CᵣₛUᵣₛUᵣₛAᵣₛAᵣₛUᵣₛUᵣₛUᵣₛCᵣₛUᵣₛAᵣₛCᵣₛUᵣₛCᵣₛUᵣₛUᵣₛGᵣₛUᵣₛAᵣₛGᵣₛAᵣₛUᵣₛCᵣₛ | 40 | 26 | 30 |
| | UᵣₛGᵣₛAᵣₛUᵣₛGᵣₛGᵣₛUᵣₛCᵣₛCᵣₛAᵣₛUᵣₛGᵣₛUᵣₛCᵣₛUᵣₛGᵣₛUᵣ | | | |
| 1086670 | CₘₛUᵣₛUᵣₛAᵣₛAᵣₛUᵣₛUᵣₛUᵣₛCᵣₛUᵣₛAᵣₛCᵣₛUᵣₛCᵣₛUᵣₛUᵣₛGᵣₛUᵣₛAᵣₛGᵣₛAᵣₛUᵣₒCᵣₒ | 40 | 68 | 30 |
| | UᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | | |
| 1086671 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 77 | 30 |
| | CᵣₒUᵣₒGᵣₒAᵣₒUᵣₒGᵣₒGᵣₛUᵣₒCᵣₛCᵣₒAᵣₛUᵣₒGᵣₛUᵣₒCᵣₛUᵣₒGₘₛUₘ | | | |

A subscript “m” indicates a 2′-O-methyl modification. A subscript “r” indicates an unmodified, 2′-hydroxy sugar moiety. A subscript “o” indicates a phosphate internucleoside linkage, and a subscript “s” indicates a phosphorothioate internucleoside linkage. The underlined portion of each crRNA is the target recognition portion, and the bolded nucleosides are linker nucleosides. The portion that is neither bold nor underlined is the CRISPR recognition portion of each crRNA. In the table above, the CRISPR recognition portions of the crRNAs recognize Cpf1. The modified crRNAs in the table above are 5′-stabilized, and the linker nucleosides comprise the 5′-stabilizing modifications.

### Example 7: Gene Editing Effects of Modified crRNAs

Modified crRNAs described below and in Examples 4, 5, and 6 were tested for their effects on gene editing of DNMT1 relative to crRNA 989549. HEK293T cells were transfected as described in Example 2, with 3 μL of 100 μM of a crRNA listed in the table below. Genomic DNA was isolated and analyzed as described in Example 1. The NHEJ incidence for each modified crRNA was normalized to the NHEJ incidence observed for crRNA 989549. The normalized values are referred to as the gene disruption percentages. The results, shown in the table below, indicate that multiple modified crRNAs edited the target gene.

TABLE 7
crRNAs targeting DNMT1

| Name | Sequence (5′ to 3′) | Length | SEQ ID No. |
|---|---|---|---|
| 1120133 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒ | 40 | 30 |
| | GᵣₒAᵣₒUᵣₒGᵣₒGᵣₒUᵣₒCᵣₒCᵣₒAᵣₒUᵣₒGᵣₒU_{fo}C_{fo}U_{fs}GₘₛUₘ | | |
| 1120139 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒ | 40 | 30 |
| | GᵣₒAᵣₒUᵣₒGᵣₒGᵣₛUᵣₒCᵣₛCᵣₒAᵣₛUᵣₒGᵣₛU_{fo}C_{fs}U_{fs}GₘₛUₘ | | |
| 1120140 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCₘₒUᵣₒ | 40 | 30 |
| | GᵣₒAᵣₒUᵣₒGᵣₒGᵣₛUₘₒCₘₛCᵣₒAᵣₛUᵣₒGᵣₛU_{fo}C_{fs}U_{fs}GₘₛUₘ | | |
| 1120141 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{fo}Uᵣₒ | 40 | 30 |
| | GᵣₒAᵣₒUᵣₒGᵣₒGᵣₛU_{fo}C_{fs}CᵣₒAᵣₛUᵣₒGᵣₛU_{fo}C_{fs}U_{fs}GₘₛUₘ | | |

A subscript “m” indicates a 2-O-methyl modification. A subscript “r” indicates an unmodified, 2-hydroxy sugar moiety. A subscript “o” indicates a phosphate internucleoside linkage, and a subscript “s” indicates a phosphorothioate internucleoside linkage. A subscript “f” indicates a 2′-F modification. The underlined portion of each crRNA is the target recognition portion, and the bolded nucleosides are linker nucleosides. The portion that is neither bold nor underlined is the CRISPR recognition portion of each crRNA. In the table above, the CRISPR recognition portions of the crRNAs recognize Cpf1. The modified crRNAs in the table above are 5′-stabilized, and the linker nucleosides comprise the 5′-stabilizing modifications.

TABLE 8a
Gene disruption

| | (Normalized) gene |
|---|---|
| Name | disruption (%) |
| 989549 | 100 |
| 1038297 | 110 |
| 1038298 | 160 |
| 1038299 | 140 |
| 1120133 | 60 |
| 1120139 | 50 |
| 991461 | 90 |
| 991462 | 90 |
| 991777 | 80 |
| 991775 | 80 |
| 991776 | 90 |
| 991783 | 130 |
| 991784 | 10 |

TABLE 8b
Gene disruption

| | (Normalized) gene |
|---|---|
| Name | disruption (%) |
| 989549 | 100 |
| 1120140 | 10 |
| 1120141 | 70 |

### Example 8: Gene Editing Effects of Modified crRNAs

Modified crRNAs comprising a target recognition portion that is complementary to DNMT1 were designed and synthesized to test their effects on gene editing of DNMT1. HEK293T cells were transfected as described in Example 2, with 3 μL of 100 μM of a crRNA listed in the table below. Genomic DNA was isolated and analyzed as described in Example 1. The NHEJ incidence for each modified crRNA in Table 9 was normalized to the NHEJ incidence observed for modified crRNA 1090626. The normalized values are referred to as the gene disruption percentages. The NHEJ incidence for each modified crRNA in Table 10 was not normalized, the absolute percentages of gene disruption observed are listed. The results, shown in the tables below, indicate that multiple modified crRNAs edited the target gene.

TABLE 9
crRNAs targeting DNMT1

| | | | Norm. gene | SEQ |
|---|---|---|---|---|
| | | | disruption | ID |
| Name | Sequence (5′ to 3′) | Length | (%) | NO. |
| 1038306 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒ | 43 | 120 | 33 |
| | AᵣₒUᵣₒGᵣₒGᵣₒUᵣₒCᵣₒCᵣₒAᵣₒUᵣₒGᵣₒUᵣₒCᵣₒUᵣₒGᵣₒUᵣₒT_{do}A_{do}C_{do}T_{do}C_{d} | | | |
| 1038307 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}UᵣₒGᵣₒ | 43 | 143 | 33 |
| | AᵣₒUᵣₒGᵣₒGᵣₒUᵣₒCᵣₒCᵣₒAᵣₒUᵣₒGᵣₒUᵣₒCᵣₒUᵣₒGᵣₒUᵣₒT_{do}A_{do}C_{do}T_{do}C_{d} | | | |
| 1038308 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒ | 43 | 135 | 34 |
| | AᵣₒUᵣₒGᵣₒGᵣₒT_{do}CᵣₒCᵣₒAᵣₒUᵣₒGᵣₒUᵣₒCᵣₒUᵣₒGᵣₒUᵣₒT_{do}A_{do}C_{do}T_{do}C_{d} | | | |
| 1038309 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒ | 43 | 169 | 33 |
| | AᵣₒUᵣₒGᵣₒGᵣₒUᵣₒC_{do}CᵣₒAᵣₒUᵣₒGᵣₒUᵣₒCᵣₒUᵣₒGᵣₒUᵣₒT_{do}A_{do}C_{do}T_{do}C_{d} | | | |
| 1038335 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}UᵣₒGᵣₒ | 43 | 198 | 34 |
| | AᵣₒUᵣₒGᵣₒGᵣₒT_{do}CᵣₒCᵣₒAᵣₒUᵣₒGᵣₒUᵣₒCᵣₒUᵣₒGᵣₒUᵣₒT_{do}A_{do}C_{do}T_{do}C_{d} | | | |
| 1038310 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}UᵣₒGᵣₒ | 43 | 181 | 33 |
| | AᵣₒUᵣₒGᵣₒGᵣₒUᵣₒC_{do}CᵣₒAᵣₒUᵣₒGᵣₒUᵣₒCᵣₒUᵣₒGᵣₒUᵣₒT_{do}A_{do}C_{do}T_{do}C_{d} | | | |
| 1038311 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒ | 43 | 181 | 34 |
| | AᵣₒUᵣₒGᵣₒGᵣₒT_{do}C_{do}CᵣₒAᵣₒUᵣₒGᵣₒUᵣₒCᵣₒUᵣₒGᵣₒUᵣₒT_{do}A_{do}C_{do}T_{do}C_{d} | | | |
| 1038312 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}UᵣₒGᵣₒ | 43 | 184 | 34 |
| | AᵣₒUᵣₒGᵣₒGᵣₒT_{do}C_{do}CᵣₒAᵣₒUᵣₒGᵣₒUᵣₒCᵣₒUᵣₒGᵣₒUᵣₒT_{do}A_{do}C_{do}T_{do}C_{d} | | | |
| 1090623 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}UᵣₒGᵣₒ | 42 | 105 | 35 |
| | AᵣₒUᵣₒGᵣₒGᵣₒT_{do}C_{do}CᵣₒAᵣₒUᵣₒGᵣₒUᵣₒCᵣₒUᵣₒGᵣₒUᵣₒT_{do}A_{do}C_{do}T_{d} | | | |
| 1090624 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}UᵣₒGᵣₒ | 41 | 120 | 36 |
| | AᵣₒUᵣₒGᵣₒGᵣₒT_{do}C_{do}CᵣₒAᵣₒUᵣₒGᵣₒUᵣₒCᵣₒUᵣₒGᵣₒUᵣₒT_{do}A_{do}C_{d} | | | |
| 1090625 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}UᵣₒGᵣₒ | 40 | 105 | 37 |
| | AᵣₒUᵣₒGᵣₒGᵣₒT_{do}C_{do}CᵣₒAᵣₒUᵣₒGᵣₒUᵣₒCᵣₒUᵣₒGᵣₒUᵣₒT_{do}A_{d} | | | |
| 1090626 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}UᵣₒGᵣₒ | 39 | 100 | 38 |
| | AᵣₒUᵣₒGᵣₒGᵣₒT_{do}C_{do}CᵣₒAᵣₒUᵣₒGᵣₒUᵣₒCᵣₒUᵣₒGᵣₒUᵣₒT_{d} | | | |
| 1090627 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}UᵣₒGᵣₒ | 38 | 11 | 39 |
| | AᵣₒUᵣₒGᵣₒGᵣₒT_{do}C_{do}CᵣₒAᵣₒUᵣₒGᵣₒUᵣₒCᵣₒUᵣₒGᵣₒT_{d} | | | |
| 1038313 | C_{do}T_{do}UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒ | 45 | 161 | 40 |
| | UᵣₒGᵣₒAᵣₒUᵣₒGᵣₒGᵣₒUᵣₒCᵣₒCᵣₒAᵣₒUᵣₒGᵣₒUᵣₒCᵣₒUᵣₒGᵣₒUᵣₒT_{do}A_{do}C_{do}T_{do}C_{d} | | | |

TABLE 10
crRNAs targeting DNMT1

| | | | Abs. Gene | SEQ |
|---|---|---|---|---|
| | | | disruption | ID |
| Name | Sequence (5′ to 3′) | Length | (%) | NO. |
| 1038313 | C_{do}T_{do}UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒ | 45 | 21 | 40 |
| | UᵣₒGᵣₒAᵣₒUᵣₒGᵣₒGᵣₒUᵣₒCᵣₒCᵣₒAᵣₒUᵣₒGᵣₒUᵣₒCᵣₒUᵣₒGᵣₒUᵣₒT_{do}A_{do}C_{do}T_{do}C_{d} | | | |
| 1090628 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒ | 43 | n.d. | 41 |
| | AᵣₒGᵣₒGᵣₒUᵣₒCᵣₒCᵣₒAᵣₒUᵣₒGᵣₒUᵣₒCᵣₒT_{do}G_{do}T_{do}T_{do}A_{do}C_{do}T_{do}C_{d} | | | |
| 1090629 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒ | 43 | n.d. | 42 |
| | AᵣₒUᵣₒGᵣₒGᵣₒUᵣₒCᵣₒC_{do}A_{do}T_{do}G_{do}T_{do}C_{do}T_{do}G_{do}T_{do}T_{do}A_{do}C_{do}T_{do}C_{d} | | | |
| 1090630 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒ | 43 | n.d. | 43 |
| | AᵣₒUᵣₒGᵣₒG_{do}T_{do}C_{do}C_{do}A_{do}T_{do}G_{do}T_{do}C_{do}T_{do}G_{do}T_{do}T_{do}A_{do}C_{do}T_{do}C_{d} | | | |
| 1090631 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒUᵣₒGᵣₒ | 43 | n.d. | 43 |
| | AᵣₒUᵣₒG_{do}G_{do}T_{do}C_{do}C_{do}A_{do}T_{do}G_{do}T_{do}C_{do}T_{do}G_{do}T_{do}T_{do}A_{do}C_{do}T_{do}C_{d} | | | |
| 1090632 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}T_{do}G_{do} | 43 | n.d. | 44 |
| | A_{do}T_{do}G_{do}G_{do}T_{do}C_{do}C_{do}A_{do}T_{do}G_{do}T_{do}C_{do}T_{do}G_{do}T_{do}T_{do}A_{do}C_{do}T_{do}C_{d} | | | |
| 1090633 | T_{do}A_{do}A_{do}T_{do}T_{do}T_{do}^{m}C_{do}T_{do}A_{do}^{m}C_{do}T_{do}^{m}C_{do}T_{do}T_{do}G_{do}T_{do}A_{do}G_{do}A_{do}T_{do}^{m}C_{do} | 43 | n.d. | 45 |
| | T_{do}G_{do}A_{do}T_{do}G_{do}G_{do}T_{do}^{m}C_{do}^{m}C_{do}A_{do}T_{do}G_{do}T_{do}^{m}C_{do}T_{do}G_{do}T_{do}T_{do}A_{do}^{m}C_{do} | | | |
| | T_{do}^{m}C_{d} | | | |

In the tables above, a subscript “r” indicates an unmodified, 2′-hydroxy sugar moiety. A subscript “d” indicates a modified, 2′-deoxy sugar moiety. A subscript “o” indicates a phosphate internucleoside linkage. A “C” following a superscript “m” indicates a 5-methyl cytosine. The underlined portion of each crRNA is the target recognition portion, and the bolded nucleosides are linker nucleosides. The portion that is neither bold nor underlined is the CRISPR recognition portion of each crRNA. The CRISPR recognition portions of the crRNAs recognize Cpf1.

### Example 9: Gene Editing Effects of Modified crRNAs

Modified crRNAs comprising a target recognition portion that is complementary to Low Density Lipoprotein Receptor (LDLR) were designed and synthesized to test their effects on gene editing of LDLR. HEK293T cells were transfected as described in Example 2, with 3 μL of 100 μM of a crRNA listed in the table below. Genomic DNA was isolated and analyzed as described in Example 1 except that the PCR primers used to amplify the crRNA target site in the LDLR gene were forward: 5′-GGAGACCCAAATACAACAAATC-3′ (SEQ ID NO: 56) and reverse: 5′-CTAGACTCCGTCTCAAAGAAG-3′ (SEQ ID NO: 57). The NHEJ incidence for each modified crRNA was normalized to the NHEJ incidence observed for crRNA 1091152. The normalized values are referred to as the gene disruption percentages. The results, shown in the table below, indicate that modified crRNAs edited the target gene.

TABLE 11a
crRNAs targeting LDLR

| | | | Norm. gene | SEQ |
|---|---|---|---|---|
| | | | disruption | ID |
| Name | Sequence (5′ to 3′) | Length | (%) | NO. |
| 1091152 | CᵣₒUᵣₒUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒ | 40 | 100 | 46 |
| | AᵣₒGᵣₒCᵣₒUᵣₒAᵣₒGᵣₒGᵣₒAᵣₒCᵣₒAᵣₒCᵣₒAᵣₒGᵣₒCᵣₒAᵣₒGᵣₒGᵣ | | | |
| 1091153 | CₘₛUᵣₒUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do} | 40 | n.d. | 46 |
| | AᵣₒGᵣₒCᵣₒUᵣₒAᵣₒGᵣₒG_{do}A_{do}CᵣₒAᵣₒCᵣₒAᵣₒGᵣₒCᵣₒAᵣₒGₘₛGₘ | | | |
| 1091154 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}AᵣₒGᵣₒ | 43 | 120 | 47 |
| | CᵣₒUᵣₒAᵣₒGᵣₒGᵣₒAᵣₒCᵣₒAᵣₒCᵣₒAᵣₒGᵣₒCᵣₒAᵣₒGᵣₒGᵣₒT_{do}C_{do}G_{do}T_{do}G_{d} | | | |
| 1091155 | UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}AᵣₒGᵣₒ | 43 | 100 | 47 |
| | CᵣₒUᵣₒAᵣₒGᵣₒG_{do}A_{do}CᵣₒAᵣₒCᵣₒAᵣₒGᵣₒCᵣₒAᵣₒGᵣₒGᵣₒT_{do}C_{do}G_{do}T_{do}G_{d} | | | |
| 1091156 | UₘₛAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}AᵣₒGᵣₒ | 43 | 120 | 48 |
| | CᵣₒUᵣₒAᵣₒGᵣₒGᵣₒAᵣₒCᵣₒAᵣₒCᵣₒAᵣₒGᵣₒCᵣₒAᵣₒGᵣₒGᵣₒUᵣₒCᵣₒGᵣₒUₘₛGₘ | | | |
| 1091157 | UₘₛAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do}AᵣₒGᵣₒ | 43 | 110 | 48 |
| | CᵣₒUᵣₒAᵣₒGᵣₒG_{do}A_{do}CᵣₒAᵣₒCᵣₒAᵣₒGᵣₒCᵣₒAᵣₒGᵣₒGᵣₒUᵣₒCᵣₒGᵣₒUₘₛGₘ | | | |
| 1091158 | C_{do}T_{do}UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do} | 45 | 120 | 49 |
| | AᵣₒGᵣₒCᵣₒUᵣₒAᵣₒGᵣₒGᵣₒAᵣₒCᵣₒAᵣₒCᵣₒAᵣₒGᵣₒCᵣₒAᵣₒGᵣₒGᵣₒT_{do}C_{do}G_{do}T_{do}G_{d} | | | |
| 1091159 | C_{do}T_{do}UᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do} | 45 | 100 | 49 |
| | AᵣₒGᵣₒCᵣₒUᵣₒAᵣₒGᵣₒG_{do}A_{do}CᵣₒAᵣₒCᵣₒAᵣₒGᵣₒCᵣₒAᵣₒGᵣₒGᵣₒT_{do}C_{do}G_{do}T_{do}G_{d} | | | |
| 1091160 | CₘₛUₘₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do} | 45 | 110 | 50 |
| | AᵣₒGᵣₒCᵣₒUᵣₒAᵣₒGᵣₒGᵣₒAᵣₒCᵣₒAᵣₒCᵣₒAᵣₒGᵣₒCᵣₒAoGᵣₒGᵣₒUᵣₒCᵣₒGᵣₒUₘₛGₘ | | | |
| 1091161 | CₘₛUᵣₒUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do} | 45 | 120 | 50 |
| | AᵣₒGᵣₒCᵣₒUᵣₒAᵣₒGᵣₒG_{do}A_{do}CᵣₒAᵣₒCᵣₒAᵣₒGᵣₒCᵣₒAᵣₒGᵣₒGᵣₒUᵣₒCᵣₒGᵣₒUₘₛGₘ | | | |

TABLE 11b
crRNAs targeting LDLR

| | | | Norm. gene | SEQ |
|---|---|---|---|---|
| | | | disruption | ID |
| Name | Sequence (5′ to 3′) | Length | (%) | NO. |
| 1091152 | CᵣₒUᵣₒUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒ | 40 | 100 | 51 |
| | AᵣₒGᵣₒCᵣₒUᵣₒAᵣₒGᵣₒGᵣₒAᵣₒCᵣₒAᵣₒCᵣₒAᵣₒGᵣₒCᵣₒAᵣₒGᵣₒGᵣ | | | |
| 1120137 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCₘₒ | 40 | 10 | 51 |
| | AᵣₒGᵣₒCᵣₒUᵣₒAᵣₒGᵣₛGₘₒAₘₛCᵣₒAᵣₛCᵣₒAᵣₛG_{fo}C_{fs}A_{fs}GₘₛGₘ | | | |
| 1120138 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{fo} | 40 | 60 | 51 |
| | AᵣₒGᵣₒCᵣₒUᵣₒAᵣₒGᵣₛG_{fo}A_{fs}CᵣₒAᵣₛCᵣₒAᵣₛG_{fo}C_{fs}A_{fs}GₘₛGₘ | | | |

A subscript “m” indicates a 2′-O-methyl modification. A subscript “r” indicates an unmodified, 2′-hydroxy sugar moiety. A subscript “d” indicates a modified, 2′-deoxy sugar moiety. A subscript “o” indicates a phosphate internucleoside linkage, and a subscript “s” indicates a phosphorothioate internucleoside linkage. The underlined portion of each crRNA is the target recognition portion, and the bolded nucleosides are linker nucleosides. The portion that is neither bold nor underlined is the CRISPR recognition portion of each crRNA. In the table above, the CRISPR recognition portions of the crRNAs recognize Cpf1.

### Example 10: Gene Editing Effects of Modified crRNAs

Modified crRNAs comprising a target recognition portion that is complementary to LDLR were designed and synthesized to test their effects on gene editing of LDLR as described in Example 9. The NHEJ incidence for each crRNA was not normalized. The values in the table below are the absolute gene disruption percentages. The results indicate that modified crRNAs edited the target gene.

TABLE 12
crRNAs targeting LDLR

| | | | Abs. Gene | SEQ |
|---|---|---|---|---|
| | | | disruption | ID |
| Name | Sequence (5′ to 3′) | Length | (%) | NO. |
| 1091152 | CᵣₒUᵣₒUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒ | 40 | 26 | 51 |
| | AᵣₒGᵣₒCᵣₒUᵣₒAᵣₒGᵣₒGᵣₒAᵣₒCᵣₒAᵣₒCᵣₒAᵣₒGᵣₒCᵣₒAᵣₒGᵣₒGᵣ | | | |
| 1091153 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{do} | 40 | 4 | 51 |
| | AᵣₒGᵣₒCᵣₒUᵣₒAᵣₒGᵣₒG_{do}A_{do}CᵣₒAᵣₒCᵣₒAᵣₒGᵣₒCᵣₒAᵣₛGₘₛGₘ | | | |
| 1091162 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒ | 40 | 18 | 51 |
| | AᵣₒGᵣₒCᵣₒUᵣₒAᵣₒGᵣₒGᵣₒAᵣₒCᵣₒAᵣₒCᵣₒAᵣₒGᵣₒCᵣₒAᵣₛGₘₛGₘ | | | |
| 1091164 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒC_{fo} | 40 | 18 | 51 |
| | AᵣₒGᵣₒCᵣₒUᵣₒAᵣₒGᵣₒG_{fo}A_{fo}CᵣₒAᵣₒCᵣₒAᵣₒGᵣₒCᵣₒAᵣₛGₘₛGₘ | | | |

A subscript “m” indicates a 2′-O-methyl modification. A subscript “r” indicates an unmodified, 2′-hydroxy sugar moiety. A subscript “d” indicates a modified, 2′-deoxy sugar moiety. A subscript “f” indicates a 2′-F modification. A subscript “o” indicates a phosphate internucleoside linkage, and a subscript “s” indicates a phosphorothioate internucleoside linkage. The underlined portion of each crRNA is the target recognition portion, and the bolded nucleosides are linker nucleosides. The portion that is neither bold nor underlined is the CRISPR recognition portion of each crRNA. In the table above, the CRISPR recognition portions of the crRNAs recognize Cpf1.

### Example 11: Gene Editing Effects of crRNAs

crRNAs comprising target recognition portions complementary to various targets were designed and synthesized to test their effects on gene editing. HEK293T cells were transfected as described in Example 2, with 3 μL of 100 μM of a crRNA listed in the table below. Genomic DNA was isolated and analyzed as described in Example 1 except that the PCR primers used to amplify the crRNA target site were one of the following: for the Complement 5 gene (C5, Table 13), forward: 5′-CATGGGGTAACCCAGCAAAC-3′ (SEQ ID NO: 58) and reverse: 5′-GGAAATAAGTGATGGGGCAGG-3′ (SEQ ID NO: 59); for the Empty Spiracles Homeobox 1 gene (EMX1, Table 14), forward: 5′-CCATCCCCTTCTGTGAATGT-3′ (SEQ ID NO: 60) and reverse: 5′-GGAGATTGGAGACACGGAGA-3′ (SEQ ID NO: 61); for the Glutamate Ionotrpoic Receptor NMDA Type Subunit 2B gene (GRIN2b, Table 15), forward: 5′-GCATACTCGCATGGCTACCT-3′ (SEQ ID NO: 62) and reverse: 5′-CTCCCTGCAGCCCCTTTTTA-3′ (SEQ ID NO: 63); for the Transthyretin gene (TTR, Table 16), forward: 5′-CAGAATCAGCAGGTTTGCAG-3′ (SEQ ID NO: 64) and reverse: 5′-CAAACCTAATGCACCAAAGC-3′ (SEQ ID NO: 65). The NHEJ incidence for each crRNA was not normalized. The values in the tables below are the absolute gene disruption percentages. The results, shown in the tables below, indicate that most crRNAs edited the corresponding target genes and that there is some variability among different targets.

TABLE 13
crRNA targeting C5

| | | | Abs. Gene | SEQ |
|---|---|---|---|---|
| | | | disruption | ID |
| Name | Sequence (5′ to 3′) | Length | (%) | NO. |
| 1091478 | CᵣₒUᵣₒUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒUᵣₒ | 40 | <5 | 52 |
| | AᵣₒCᵣₒUᵣₒCᵣₒCᵣₒAᵣₒGᵣₒAᵣₒCᵣₒCᵣₒAᵣₒGᵣₒUᵣₒCᵣₒAᵣₒGᵣₒGᵣ | | | |

TABLE 14
crRNA targeting EMX1

| | | | Abs. Gene | SEQ |
|---|---|---|---|---|
| | | | disruption | ID |
| Name | Sequence (5′ to 3′) | Length | (%) | NO. |
| 1091480 | CᵣₒUᵣₒUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒUᵣₒ | 40 | 16 | 53 |
| | GᵣₒGᵣₒUᵣₒUᵣₒGᵣₒCᵣₒCᵣₒCᵣₒAᵣₒCᵣₒCᵣₒCᵣₒUᵣₒAᵣₒGᵣₒUᵣₒCᵣ | | | |

TABLE 15
crRNA targeting GRIN2b

| | | | Abs. Gene | SEQ |
|---|---|---|---|---|
| | | | disruption | ID |
| Name | Sequence (5′ to 3′) | Length | (%) | NO. |
| 1091484 | CᵣₒUᵣₒUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒGᵣₒ | 40 | 19 | 54 |
| | UᵣₒGᵣₒCᵣₒUᵣₒCᵣₒAᵣₒAᵣₒUᵣₒGᵣₒAᵣₒAᵣₒAᵣₒGᵣₒGᵣₒAᵣₒGᵣₒAᵣ | | | |

TABLE 16
crRNA targeting TTR

| | | | Abs. Gene | SEQ |
|---|---|---|---|---|
| | | | disruption | ID |
| Name | Sequence (5′ to 3′) | Length | (%) | NO. |
| 1091482 | CᵣₒUᵣₒUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒUᵣₒ | 40 | n.d. | 55 |
| | GᵣₒUᵣₒCᵣₒUᵣₒGᵣₒAᵣₒGᵣₒGᵣₒCᵣₒUᵣₒGᵣₒGᵣₒCᵣₒCᵣₒCᵣₒUᵣₒAᵣ | | | |

The legend for Table 12 applies to Tables 13-16.

### Example 12: Gene Editing Effects of Modified crRNAs

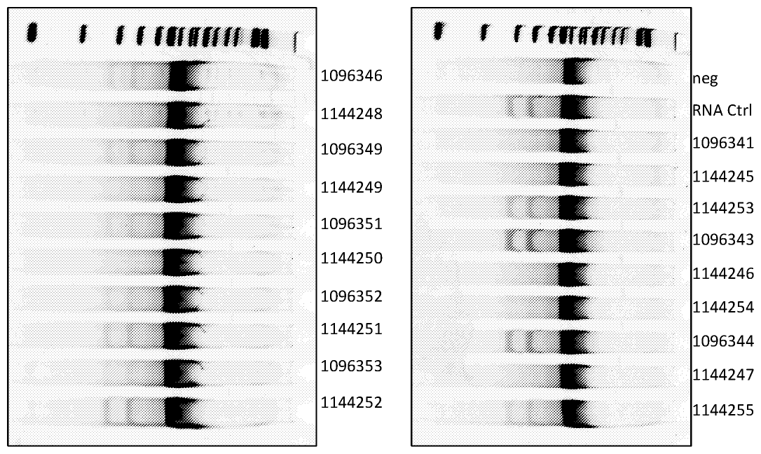

Modified crRNAs comprising a target recognition portion that is complementary to DNMT1 were designed and synthesized to test their effects on gene editing of DNMT1 relative to unmodified crRNA 989549 (see Table 6). HEK293T cells were transfected as described in Example 2, with 3 μL of 100 μM of a modified crRNA listed in the table below, crRNA 989549 (“RNA Ctrl”), or no crRNA (“neg”). Genomic DNA was isolated and analyzed as described in Example 1 except that NHEJ incidence was not quantified. The resulting DNA gel is shown in FIG. 1 and indicates that multiple modified crRNAs comprising at least one modified sugar moiety in the CRISPR recognition portion edited the target gene.

TABLE 17
crRNAs targeting DNMT1

| | | | SEQ ID |
|---|---|---|---|
| Name | Sequence (5′ to 3′) | Length | NO. |
| 1096341 | CₘₛUᵣₛUᵣₒAᵣₒAᵣₒUᵣₒUᵣₒUᵣₒ^{m}CₖₒUᵣₒAᵣₒCᵣₒUᵣₒCᵣₒUᵣₒUᵣₒGᵣₒUᵣₒAᵣₒGᵣₒAᵣₒUᵣₒ | 40 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1096343 | CₘₛUᵣₛUᵣₒAᵣₒAᵣₒUᵣₒUᵣₒUᵣₒCᵣₒUᵣₒAₖₒCᵣₒUᵣₒCᵣₒUᵣₒUᵣₒGᵣₒUᵣₒAᵣₒGᵣₒAᵣₒUᵣₒ | 40 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1096344 | CₘₛUᵣₛUᵣₒAᵣₒAᵣₒUᵣₒUᵣₒUᵣₒCᵣₒUᵣₒAᵣₒ^{m}CₖₒUᵣₒCᵣₒUᵣₒUᵣₒGᵣₒUᵣₒAᵣₒGᵣₒAᵣₒUᵣₒ | 40 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1096346 | CₘₛUᵣₛUᵣₒAᵣₒAᵣₒUᵣₒUᵣₒUᵣₒCᵣₒUᵣₒAᵣₒCᵣₒUᵣₒ^{m}CₖₒUᵣₒUᵣₒGᵣₒUᵣₒAᵣₒGᵣₒAᵣₒUᵣₒ | 40 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1096349 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGₖₒUᵣₒAᵣₒGᵣₒAᵣₒUᵣₒ | 40 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1096351 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAₖₒGᵣₒAᵣₒUᵣₒ | 40 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1096352 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGₖₒAᵣₒUᵣₒ | 40 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1096353 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAₖₒUᵣₒ | 40 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1144245 | CₘₛUᵣₛUᵣₒAᵣₒAₖₒUᵣₒUᵣₒUᵣₒCᵣₒUᵣₒAᵣₒCᵣₒTₖₒCᵣₒUᵣₒUᵣₒGᵣₒUᵣₒAᵣₒGᵣₒAᵣₒUᵣₒ | 40 | 71 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1144246 | CₘₛUᵣₛUᵣₒAᵣₒAᵣₒTₖₒUᵣₒUᵣₒCᵣₒTₖₒAᵣₒCᵣₒUᵣₒCᵣₒUᵣₒUᵣₒGᵣₒUᵣₒAᵣₒGᵣₒAᵣₒUᵣₒ | 40 | 72 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1144247 | CₘₛUᵣₛUᵣₒAᵣₒAᵣₒTₖₒUᵣₒUᵣₒCᵣₒUᵣₒAₖₒCᵣₒUᵣₒCᵣₒUᵣₒUᵣₒGᵣₒUᵣₒAᵣₒGᵣₒAᵣₒUᵣₒ | 40 | 73 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1144248 | CₘₛUᵣₛUᵣₒAᵣₒAᵣₒUᵣₒTₖₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 74 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1144249 | CₘₛUᵣₛUᵣₒAᵣₒAᵣₒUᵣₒUᵣₒTₖₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 75 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1144250 | CₘₛUᵣₛUᵣₒAᵣₒAᵣₒUᵣₒUᵣₒUᵣₒCᵣₒUᵣₒAᵣₒCᵣₒTₖₒCᵣₒUᵣₒUᵣₒGᵣₒUᵣₒAᵣₒGᵣₒAᵣₒUᵣ | 40 | 71 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1144251 | CₘₛUᵣₛUᵣₒAᵣₒAᵣₒUᵣₒUᵣₒUᵣₒCᵣₒUᵣₒAᵣₒCᵣₒUᵣₒCᵣₒTₖₒUᵣₒGᵣₒUᵣₒAᵣₒGᵣₒAᵣₒUᵣₒCᵣₒ | 40 | 76 |
| | UᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1144252 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒTₖₒGᵣₒUᵣₒAᵣₒGᵣₒAᵣₒUᵣₒCᵣₒ | 40 | 77 |
| | UᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1144253 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒTₖₒAᵣₒGᵣₒAᵣₒUᵣₒCᵣₒ | 40 | 78 |
| | UᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1144254 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒTₖₒCᵣₒ | 40 | 79 |
| | UᵣₒGᵣₒAᵣₒUᵣₒGᵣₒGᵣₒUᵣₒCᵣₒCᵣₒAᵣₒUᵣₒGᵣₒUᵣₒCᵣₒUᵣGₘₛUₘ | | |
| 1144255 | CₘₛUᵣₛUᵣₒAᵣₒAᵣₒUᵣₒUᵣₒUᵣₒCᵣₒTₖₒAᵣₒCᵣₒUᵣₒCᵣₒUᵣₒUᵣₒGᵣₒUᵣₒAᵣₒGᵣₒAᵣₒUᵣₒCᵣₒ | 40 | 80 |
| | UᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |

A subscript “m” indicates a 2′-O-methyl modification. A subscript “r” indicates an unmodified, 2′-hydroxy sugar moiety. A subscript “k” indicates a cEt modification. A subscript “o” indicates a phosphate internucleoside linkage, and a subscript “s” indicates a phosphorothioate internucleoside linkage. The underlined portion of each crRNA is the target recognition portion, and the bolded nucleosides are linker nucleosides. The portion that is neither bold nor underlined is the CRISPR recognition portion of each crRNA. In the table above, the CRISPR recognition portions of the crRNAs recognize Cpf1.

### Example 13: Gene Editing Effects of Modified crRNAs

Modified crRNAs comprising a target recognition portion that is complementary to DNMT1 were designed to test their effects on gene editing of DNMT1. HEK293T cells are transfected as described in Example 2. Genomic DNA is isolated and analyzed as described in Example 1.

TABLE 18
crRNAs targeting DNMT1

| | | | SEQ ID |
|---|---|---|---|
| Name | Sequence (5′ to 3′) | Length | NO. |
| 1186843 | CₘₛUᵣₛUᵣₒAᵣₒAₖₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒCᵣₒ | 40 | 30 |
| | UᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1186844 | CₘₛUᵣₛUᵣₒAᵣₒAᵣₒUᵣₒUᵣₒUᵣₒCᵣₒUᵣₒAₖₒ^{m}CₖₒUᵣₒCᵣₒUᵣₒUᵣₒGᵣₒUᵣₒAᵣₒGᵣₒAᵣₒUᵣₒ | 40 | 30 |
| | CᵣₒUᵣₒGᵣₒAᵣₒUᵣₒGᵣₒGᵣₒUᵣₒCᵣₒCᵣₒAᵣₒUᵣₒGᵣₒUᵣₒCᵣₒUᵣₛGₘₛUₘ | | |
| 1186845 | CₘₛUᵣₛUᵣₒAᵣₒAᵣₒUᵣₒUᵣₒUᵣₒCᵣₒUᵣₒAₖₒCᵣₒUᵣₒCᵣₒUᵣₒUᵣₒGᵣₒUᵣₒAᵣₒGᵣₒAᵣₒUᵣₒ | 40 | 30 |
| | C_{fo}UᵣₒGᵣₒAᵣₒUᵣₒGᵣₒGᵣₛU_{fo}C_{fs}CᵣₒAᵣₛUᵣₒGᵣₛU_{fo}C_{fs}U_{fs}GₘₛUₘ | | |
| 1186846 | CₘₛUᵣₛUᵣₒAᵣₒAᵣₒUᵣₒUᵣₒUᵣₒCᵣₒUᵣₒAᵣₒ^{m}CₖₒUᵣₒCᵣₒUᵣₒUᵣₒGᵣₒUᵣₒAᵣₒGᵣₒAᵣₒUᵣₒ | 40 | 30 |
| | C_{fo}UᵣₒGᵣₒAᵣₒUᵣₒGᵣₒGᵣₛU_{fo}C_{fs}CᵣₒAᵣₛUᵣₒGᵣₛU_{fo}C_{fs}U_{fs}GₘₛUₘ | | |
| 1186847 | CₘₛUᵣₛUᵣₒAᵣₒAᵣₒUᵣₒUᵣₒUᵣₒCᵣₒUᵣₒAₖₒ^{m}CₖₒUᵣₒCᵣₒUᵣₒUᵣₒGᵣₒUᵣₒAᵣₒGᵣₒAᵣₒUᵣₒ | 40 | 30 |
| | C_{fo}UᵣₒGᵣₒAᵣₒUᵣₒGᵣₒGᵣₛU_{fo}C_{fs}CᵣₒAᵣₛUᵣₒGᵣₛU_{fo}C_{fs}U_{fs}GₘₛUₘ | | |
| 1186848 | CₘₛUᵣₛUᵣₒAᵣₒAᵣₒUᵣₒUᵣₒUᵣₒCᵣₒUᵣₒAᵣₒCᵣₒUᵣₒCᵣₒUᵣₒUᵣₒGᵣₒUᵣₒAᵣₒGᵣₒAᵣₒUᵣₒ | 40 | 81 |
| | C_{do}UᵣₒGᵣₒAᵣₒUᵣₒGᵣₒGᵣₒT_{do}C_{do}CᵣₒAᵣₛUᵣₒGᵣₒT_{do}C_{do}T_{do}GₘₛUₘ | | |

A subscript “m” indicates a 2′-O-methyl modification. A subscript “r” indicates an unmodified, 2′-hydroxy sugar moiety. A subscript “d” indicates a modified, 2′-deoxy sugar moiety. A subscript “k” indicates a cEt modification. A subscript “f” indicates a 2′-F modification. A subscript “o” indicates a phosphate internucleoside linkage, and a subscript “s” indicates a phosphorothioate internucleoside linkage. The underlined portion of each crRNA is the target recognition portion, and the bolded nucleosides are linker nucleosides. The portion that is neither bold nor underlined is the CRISPR recognition portion of each crRNA. In the table above, the CRISPR recognition portions of the crRNAs recognize Cpf1.

1. A compound comprising a modified crRNA consisting of 35-45 linked nucleosides.

2. A compound comprising a modified crRNA, wherein the CRISPR recognition portion of the modified crRNA consists of 17-20 linked nucleosides.

3. A compound comprising a modified crRNA, wherein the target recognition portion of the modified crRNA consists of 18-23 linked nucleosides.

4. A compound comprising a modified crRNA, wherein the modified crRNA comprises at least one linker nucleoside.

5. A compound comprising a 5′-stabilized modified crRNA.

6. The compound of any of claims 1-5, wherein the compound comprises a stabilizing conjugate group.

7. The compound of any of claims 1-5, wherein the crRNA comprises at least one linker nucleoside comprising a stabilizing modification.

8. The compound of any of claims 1-4, wherein the modified crRNA is 5′-stabilized.

9. The compound of any of claims 1-8, wherein the modified crRNA is 3′-stabilized.

10. The compound of any of claims 1-9, wherein the CRISPR recognition portion of the modified crRNA binds to a Cpf1 nuclease.

11. The compound of any of claims 1-10, wherein the target recognition portion of the modified crRNA comprises at least one modification that increases affinity of the crRNA for a target DNA or RNA.

12. The compound of any of claims 10-11, wherein the CRISPR recognition portion of the modified crRNA comprises at least one modification that increases affinity of the crRNA for a Cpf1 nuclease.

13. The compound of any of claims 1-12, wherein at least one nucleobase of the modified crRNA is thymine.

14. The compound of any of claims 1-13, wherein at least one nucleobase of the modified crRNA is a modified nucleobase.

15. The compound of claim 14, wherein the modified nucleobase is 5-methyl cytosine.

16. The compound of any of claims 1-15, wherein modified crRNA consists of 35-42 linked nucleosides.

17. The compound of any of claim 1-15, wherein the modified crRNA consists of 36-40 linked nucleosides.

18. The compound of any of claims 1-17, wherein the modified crRNA comprises at least two linker nucleosides.

19. The compound of claim 18, wherein at least two linker nucleosides are linked to the CRISPR recognition portion of the modified crRNA.

20. The compound of claim 19, wherein at least two linker nucleosides are linked to the 5′-end of the CRISPR recognition portion of the modified crRNA.

21. The compound of any of claims 1-20, wherein the CRISPR recognition portion of the modified crRNA consists of 18-20 linked nucleosides.

22. The compound of claim 21, wherein the CRISPR recognition portion of the modified crRNA consists of 18 linked nucleosides.

23. The compound of claim 21, wherein the CRISPR recognition portion of the modified crRNA consists of 19 linked nucleosides.

24. The compound of claim 21, wherein the CRISPR recognition portion of the modified crRNA consists of 20 linked nucleosides.

25. The compound of any of claims 1-24, wherein the target recognition protion of the modified crRNA consists of 18-22 linked nucleosides.

26. The compound of any of claims 1-24, wherein the target recognition protion of the modified crRNA consists of 18-20 linked nucleosides.

27. The compound of claim 26, wherein the target recognition protion of the modified crRNA consists of 18 linked nucleosides.

28. The compound of claim 26, wherein the target recognition protion of the modified crRNA consists of 19 linked nucleosides.

29. The compound of claim 26, wherein the target recognition protion of the modified crRNA consists of 20 linked nucleosides.

30. The compound of any of claims 1-29, wherein at least one internucleoside linkage of the modified crRNA is a modified internucleoside linkage.

31. The compound of claim 30, wherein at least one internucleoside linkage is a phosphorothioate internucleoside linkage.

32. The compound of claim 30 or 31, wherein each internucleoside linkage of the modified crRNA is a modified internucleoside linkage.

33. The compound of any of claims 30-32, wherein at least one internucleoside linkage is a neutral internucleoside linkage.

34. The compound of claim 33, wherein at least one modified internucleoside linkage comprises a methoxypropyl group.

35. The compound of claim 33, wherein at least one modified internucleoside linkage comprises a phosphonoacetate.

36. The compound of claim 33, wherein at least one modified internucleoside linkage comprises a methylphosphonate.

37. The compound of any of claims 1-31, wherein each internucleoside linkage of the modified crRNA is a phosphodiester internucleoside linkage or a phosphorothioate internucleoside linkage.

38. The compound of any of claim 30, 31, or 33-37, wherein at least two internucleoside linkages of the modified crRNA are modified internucleoside linkages.

39. The compound of claim 38, wherein at least two modified internucleoside linkages of the modified crRNA are the same as one another.

40. The compound of any of claims 1-39, wherein the modified crRNA comprises one to five contiguous phosphorothioate internucleoside linkages at the 5′-end of the modified crRNA.

41. The compound of claim 40, wherein the modified crRNA comprises one phosphorothioate internucleoside linkage at the 5′-end of the modified crRNA.

42. The compound of claim 40, wherein the modified crRNA comprises two contiguous phosphorothioate internucleoside linkages at the 5′-end of the modified crRNA.

43. The compound of any of claims 1-42, wherein the modified crRNA comprises at least one linker nucleoside that is linked to the CRISPR recognition portion of the modified crRNA by a modified internucleoside linkage.

44. The compound of claim 43, wherein the modified internucleoside linkage that links the at least one linker nucleoside to the CRISPR recognition portion of the modified crRNA is a phosphorothiaote internucleoside linkage.

45. The compound of claim 44, wherein the modified crRNA comprises two linker nucleosides.

46. The compound of claim 45, wherein the linker nucleosides are linked to each other by a modified internucleoside linkage.

47. The compound of claim 46, wherein the modified internucleoside that links the linker nucleosides to each other is a phosphorothioate internucleoside linkage.

48. The compound of any claims 43-44, wherein the modified crRNA comprises more than two linker nucleosides.

49. The compound of any of claims 1-48, wherein the modified crRNA comprises one to six modified internucleoside linkages within the target recognition portion of the modified crRNA.

50. The compound of claim 49, wherein the one to six modified internucleoside linkages within the target recognition portion of the modified crRNA are contiguous.

51. The compound of claim 49, wherein the one to six modified internucleoside linkages within the target recognition portion of the modified crRNA alternate with unmodified internucleoside linkages.

52. The compound of any of claims 49-51, wherein the 3′-end of the target recognition portion of the modified crRNA contains the one to six modified internucleoside linkages.

53. The compound of any of claims 50-52, wherein the target recognition portion of the modified crRNA comprises one modified internucleoside linkage.

54. The compound of any of claims 50-52, wherein the target recognition portion of the modified crRNA comprises two modified internucleoside linkages.

55. The compound of any of claims 50-52, wherein the target recognition portion of the modified crRNA comprises three modified internucleoside linkages.

56. The compound of any of claims 50-52, wherein the target recognition portion of the modified crRNA comprises four modified internucleoside linkages.

57. The compound of any of claims 50-52, wherein the target recognition portion of the modified crRNA comprises five modified internucleoside linkages.

58. The compound of any of claims 50-52, wherein the target recognition portion of the modified crRNA comprises six modified internucleoside linkages.

59. The compound of any of claims 49-58, wherein at least one internucleoside linkage within the target recognition portion of the modified crRNA is a phosphorothioate internucleoside linkage.

60. The compound of any of claims 49-58, wherein all of the modified internucleoside linkages within the target recognition portion of the modified crRNA are phosphorothioate internucleoside linkages.

61. The compound of any of claims 1-60, wherein the target recognition portion of the modified crRNA is directly or indirectly linked to the 3′ end of the CRISPR recognition portion of the modified crRNA.

62. The compound of any of claims 1-61, wherein at least one nucleoside of the modified crRNA comprises a modified sugar moiety.

63. The compound of claim 62, wherein the 5′-terminal nucleoside of the crRNA comprises a modified sugar moiety.

64. The compound of claim 63, wherein the 5′-terminal nucleoside comprises a linearly modified sugar moiety.

65. The compound of claim 64, wherein the 5′-terminal nucleoside comprises a 2′-modified sugar moiety.

66. The compound of claim 63, wherein the 5′-terminal nucleoside comprises a bicyclic sugar moiety.

67. The compound of claim 63, wherein the 5′-terminal nucleoside comprises a modified sugar moiety selected from among: 2′-O-methyl, 2′-MOE, 2′-F, cEt, and LNA.

68. The compound of any of claims 1-67, wherein the 5′-terminal nucleoside is a linker nucleoside.

69. The compound of any of claims 62-68, wherein the 5^{th} nucleoside from the 5′-end of the CRISPR recognition portion comprises a modified sugar moiety.

70. The compound of any of claims 62-69, wherein the 6^{th} nucleoside from the 5′-end of the CRISPR recognition portion comprises a modified sugar moiety.

71. The compound of any of claims 62-70, wherein the 7^{th} nucleoside from the 5′-end of the CRISPR recognition portion comprises a modified sugar moiety.

72. The compound of any of claims 62-71, wherein the 10^{th} nucleoside from the 5′-end of the CRISPR recognition portion comprises a modified sugar moiety.

73. The compound of any of claims 62-72, wherein the 14^{th} nucleoside from the 5′-end of the CRISPR recognition portion comprises a modified sugar moiety.

74. The compound of any of claims 62-73, wherein the 1st nucleoside from the 3′-end of the CRISPR recognition portion comprises a modified sugar moiety.

75. The compound of any of claims 69-74, wherein at least one modified sugar moiety is selected from among: 2′-O-methyl, 2′-MOE, 2′-F, cEt, and LNA.

76. The compound of any of claims 69-74, wherein each modified sugar moiety is independently selected from among: 2′-O-methyl, 2′-MOE, 2′-F, cEt, and LNA.

77. The compound of claim 62, wherein the 3′-terminal nucleoside of the modified crRNA comprises a modified sugar moiety.

78. The compound of claim 77, wherein the 3′-terminal nucleoside comprises a linearly modified sugar moiety.

79. The compound of claim 78, wherein the 3′-terminal nucleoside comprises a 2′-modified sugar moiety.

80. The compound of claim 77, wherein the 3′-terminal nucleoside comprises a bicyclic sugar moiety.

81. The compound of claim 77, wherein the 3′-terminal nucleoside comprises a modified sugar moiety selected from among: 2′-O-methyl, 2′-MOE, 2′-F, cEt, and LNA.

82. The compound of any of claims 62-81, wherein the 1st nucleoside from the 5′-end of the target recognition portion comprises a modified sugar moiety.

83. The compound of any of claims 62-82, wherein the 8th nucleoside from the 5′-end of the target recognition portion comprises a modified sugar moiety.

84. The compound of any of claims 62-83, wherein the 9th nucleoside from the 5′-end of the target recognition portion comprises a modified sugar moiety.

85. The compound of any of claims 62-84, wherein one to five 3′-terminal nucleosides of the target recognition portion of the modified crRNA each comprise a modified sugar moiety.

86. The compound of claim 85, wherein the one to five 3′-terminal nucleosides of the target recognition portion of the modified crRNA each comprise the same modified sugar moiety.

87. The compound of claim 84 or 85, wherein the modified sugar moieties of the one to five 3′-terminal nucleosides of the target recognition portion are each independently selected from among 2′-O-methyl, 2′-MOE, 2′-F, cEt, and LNA.

88. The compound of any of claims 1-87, wherein the target recognition portion of the modified crRNA comprises at least one unmodified sugar moiety.

89. The compound of any of claims 1-88, wherein the CRISPR recognition portion of the modified crRNA comprises at least one unmodified sugar moiety.

90. The compound of any of claims 1-89, wherein the modified crRNA comprises at least one linker nucleoside that comprises an unmodified sugar moiety.

91. The compound of any of claims 1-90, wherein the compound consists of the modified crRNA.

92. The compound of any of claims 1-91, wherein the nucleobase sequence of the target recognition portion of the modified crRNA is at least 90% complementary to a target DNA or RNA.

93. The compound of claim 92, wherein the nucleobase sequence of the target recognition portion of the modified crRNA is 100% complementary to a target DNA or RNA.

94. The compound of any of claims 1-93, wherein the modified crRNA comprises a self-complementary region.

95. The compound of claim 94, wherein the self-complementary region is within the CRISPR recognition portion of the modified crRNA.

96. The compound of claim 94 or 95, wherein the self-complementary region can form a hairpin.

97. The compound of any of claims 94-96, wherein the self-complementary region comprises at least one modification that increases the stability of the self-complementary region.

98. The compound of any of claims 94-97, wherein the self-complementary region comprises at least one modification that increases the hybridization affinity of the self-complementary region.

99. The compound of any of claims 1-98, wherein the nucleobase sequence of the CRISPR recognition portion of the modified crRNA comprises at least 12 contiguous nucleobases of a sequence selected from Table A.

100. The compound of any of claims 1-98, wherein the nucleobase sequence of the CRISPR recognition portion of the modified crRNA consists of a sequence or a portion of a sequence selected from Table A.

101. The compound of any of claims 1-100, wherein the nucleobase sequence of the CRISPR recognition portion of the modified crRNA comprises the sequence XCXACX, wherein each X is, independently, a U nucleobase or a T nucleobase.

102. The compound of any of claims 1-100, wherein the nucleobase sequence of the CRISPR recognition portion of the modified crRNA comprises the sequence GXAGAX, wherein each X is, independently, a U nucleobase or a T nucleobase.

103. The compound of any of claims 1-100, wherein the nucleobase sequence of the CRISPR recognition portion of the modified crRNA comprises the sequence XCXACX and the sequence GXAGAX, wherein each X is, independently, a U nucleobase or a T nucleobase.

104. The compound of any of claim 1-90 or 92-103, wherein the compound comprises a conjugate group.

105. The compound of claim 104, wherein the conjugate group comprises GalNAc.

106. The compound of claim 104, wherein the conjugate group is lipophilic.

107. The compound of claim 106, wherein the conjugate group comprises a lipid.

108. A pharmaceutical composition comprising the compound of any of claims 1-107.

109. A method comprising contacting a cell with the compound or composition of any of claims 1-108.

110. The method of claim 109, wherein the cell expresses a Cpf1 nuclease.

111. A method comprising contacting a cell with the compound or composition of any of claims 1-108 and a plasmid that encodes a Cpf1 nuclease.

112. A method comprising contacting a cell with the compound or composition of any of claims 1-108 and an mRNA that encodes a Cpf1 nuclease.

113. The method of any of claims 109-112, wherein the modified crRNA is taken up by the cell in the absence of a transfection reagent.

114. The method of any of claims 109-113, wherein the cell is in an animal.

115. A method comprising administering to an animal the compound or composition of any of claims 1-108.

116. The method of claim 115, wherein the administration is subcutaneous.

117. The method of claim 115, wherein the administration is intrathecal.

118. The method of any of claims 115-117 comprising administering a plasmid that encodes a Cpf1 nuclease.

119. The method of any of claims 115-117 wherein the animal expresses a Cpf1 nuclease.

120. The method of claim 111 or 118, wherein the plasmid is delivered to cells within the animal via an adeno-associated virus (AAV).

121. The method of claim 111 or 118, wherein the plasmid is delivered to cells within the animal via a lentivirus.

122. The method of any of claims 109-121, wherein a target gene is edited.

123. The method of claim 122, wherein the modified crRNA is degraded in a cell after the target gene is edited in the cell.

124. The method of any of claim 110-112 or 118-123, wherein the Cpf1 nuclease does not exhibit nuclease activity in the absence of the modified crRNA.

125. The method of any of claims 109-124 comprising contacting the cell with a second compound that degrades or inhibits the activity or expression of the modified crRNA or a Cpf1 nuclease.

126. The method of claim 125, wherein the cell is contacted with the second compound after a target gene has been edited.

127. The method of claim 125 or 126, wherein the second compound comprises an oligonucleotide that is complementary to the modified crRNA.

128. The method of claim 125 or 126, wherein the second compound comprises a crRNA that targets a Cpf1 nuclease gene.

129. The method of claim 125 or 126, wherein the second compound comprises an oligonucleotide that is complementary to a Cpf1 transcript.

130. The method of claim 128 or 129, wherein the expression of the Cpf1 nuclease is inhibited.

131. The method of any of claims 114-130, wherein the animal is a human.

132. The method of any of claims 109-131, wherein editing of at least one off-target gene is reduced relative to editing the at least one off-target gene when unmodified crRNA or a compound comprising more than 45 nucleosides is used in place of the modified crRNA.

133. The method of any of claim 115 or 118-132, wherein the administration is intravitreal.

134. The method of any of claims 109-113, wherein the cell is a plant cell.

135. The method of any of claims 109-114, wherein the cell is a T-cell.

136. A method of treating a disease in an individual comprising administering the compound of any of claims 1-107 or the composition of claim 108 to the individual, thereby treating the disease in the individual.

137. Use of the compound of any of claims 1-107 or the composition of claim 108 for the treatment of a disease.

138. Use of the compound of any of claims 1-107 or the composition of claim 108 for preparation of a medicament.

139. A method of administering the compound of any of claims 1-107 or the composition of claim 108 to an animal, and harvesting an organ from the animal for transplantation into a human.

140. The compound of any of claims 90-107, wherein the 5′-terminal nucleoside comprises a cEt modified sugar moiety.

141. The compound of claim 140, wherein the 3′-end of the target recognition portion of the modified crRNA contains two contiguous phosphorothioate internucleoside linkages.

142. The compound of any of claims 140-141, wherein each internucleoside linkage of the CRISPR recognition portion of the modified crRNA is phosphorothioate.

143. The compound of any of claims 140-142, wherein the two 3′-terminal nucleosides of the target recognition portion of the modified crRNA each comprise a 2′-O-methyl modified sugar moiety.

144. The compound of any of claims 140-143, wherein the 1^{st} nucleoside from the 5′-end of the CRISPR recognition portion comprises an unmodified sugar moiety.

145. The compound of any of claims 140-144, wherein the modified crRNA comprises 30-38 unmodified sugar moieties.

146. The compound of claim 145, wherein the modified crRNA comprises 36 unmodified sugar moieties.

147. A pharmaceutical composition comprising the compound of any of claims 140-146.

148. A method comprising contacting a cell with the compound or composition of any of claims 140-147.

149. The method of claim 148, wherein the cell expresses a Cpf1 nuclease.

150. A method comprising contacting a cell with the compound or composition of any of claims 140-147 and a plasmid that encodes a Cpf1 nuclease.

151. A method comprising contacting a cell with the compound or composition of any of claims 140-147 and an mRNA that encodes a Cpf1 nuclease.

152. The method of any of claims 148-151, wherein the modified crRNA is taken up by the cell in the absence of a transfection reagent.

153. The method of any of claims 148-152, wherein the cell is in an animal.

154. A method comprising administering to an animal the compound or composition of any of claims 140-147.

155. The method of claim 154, wherein the administration is subcutaneous.

156. The method of claim 154, wherein the administration is intrathecal.

157. The method of any of claims 154-156 comprising administering a plasmid that encodes a Cpf1 nuclease.

158. The method of any of claims 154-156 wherein the animal expresses a Cpf1 nuclease.

159. The method of claim 150 or 157, wherein the plasmid is delivered to cells within the animal via an adeno-associated virus (AAV).

160. The method of claim 150 or 157, wherein the plasmid is delivered to cells within the animal via a lentivirus.

161. The method of any of claims 148-160, wherein a target gene is edited.

162. The method of claim 161, wherein the modified crRNA is degraded in a cell after the target gene is edited in the cell.

163. The method of any of claim 149-151 or 157-162, wherein the Cpf1 nuclease does not exhibit nuclease activity in the absence of the modified crRNA.

164. The method of any of claims 148-163 comprising contacting the cell with a second compound that degrades or inhibits the activity or expression of the modified crRNA or a Cpf1 nuclease.

165. The method of claim 164, wherein the cell is contacted with the second compound after a target gene has been edited.

166. The method of claim 164 or 165, wherein the second compound comprises an oligonucleotide that is complementary to the modified crRNA.

167. The method of claim 164 or 165, wherein the second compound comprises a crRNA that targets a Cpf1 nuclease gene.

168. The method of claim 164 or 165, wherein the second compound comprises an oligonucleotide that is complementary to a Cpf1 transcript.

169. The method of claim 167 or 168, wherein the expression of the Cpf1 nuclease is inhibited.

170. The method of any of claims 153-169, wherein the animal is a human.

171. The method of any of claims 148-170, wherein editing of at least one off-target gene is reduced relative to editing the at least one off-target gene when unmodified crRNA or a compound comprising more than 45 nucleosides is used in place of the modified crRNA.

172. The method of any of claim 154 or 157-171, wherein the administration is intravitreal.

173. The method of any of claims 148-152, wherein the cell is a plant cell.

174. The method of any of claims 148-153, wherein the cell is a T-cell.

175. A method of treating a disease in an individual comprising administering the compound of any of claims 140-146 or the composition of claim 147 to the individual.

176. A method of treating a disease in an individual comprising administering the compound of any of claims 140-146 or the composition of claim 147 to the individual, thereby treating the disease in the individual.

177. Use of the compound of any of claims 140-146 or the composition of claim 147 for the treatment of a disease.

178. Use of the compound of any of claims 140-146 or the composition of claim 147 for preparation of a medicament.

179. A method of administering the compound of any of claims 140-146 or the composition of claim 147 to an animal, and harvesting an organ from the animal for transplantation into a human.

180. The compound of any of claim 91-107 or 140-146, wherein at least one modified nucleoside of the modified crRNA is a 2′-deoxynucleoside.

181. The compound of any of claim 91-107 or 140-146, wherein at least one modified nucleoside of the modified crRNA comprises a linearly modified sugar moiety having a 2′-H substitution.

182. The compound of any of claim 91-107 or 140-146, wherein at least one modified nucleoside of the modified crRNA comprises a modified 2′-H(H) sugar moiety as found in naturally occurring DNA.

183. The compound of any of claim 91-107, 140-146, or 180-182, wherein the modified crRNA consists of 40 linked nucleosides.

184. The compound of any of claim 91-107, 140-146, or 180-182, wherein the modified crRNA consists of 43 linked nucleosides.

185. The compound of any of claim 91-107, 140-146, or 180-182, wherein the modified crRNA consists of 45 linked nucleosides.

186. The compound of any of claim 91-107, 140-146, or 180-185, wherein the target recognition portion of the modified crRNA is at least 90% complementary to a DNMT1 nucleic acid.

187. The compound of claim 186, wherein the target recognition portion is 100% complementary to a DNMT1 nucleic acid.

188. The compound of claim 186 or 187, wherein the DNMT1 nucleic acid is a deoxyribonucleic acid.

189. The compound of claim 188, wherein the DNMT1 nucleic acid is a human deoxyribonucleic acid.

190. The compound of any of claim 91-107, 140-146, or 180-185, wherein the target recognition portion of the modified crRNA is at least 90% complementary to a LDLR nucleic acid.

191. The compound of claim 190, wherein the target recognition portion is 100% complementary to a LDLR nucleic acid. The compound of claim 190 or 191, wherein the LDLR nucleic acid is a deoxyribonucleic acid.

192. The compound of claim 191, wherein the LDLR nucleic acid is a human deoxyribonucleic acid.

193. The compound of any of claim 91-107, 140-146, or 180-192, wherein the two 3′-terminal nucleosides of the modified crRNA comprise independently selected modified sugar moieities.

194. The compound of any of claim 91-107, 140-146, or 180-192, wherein the three 3′-terminal nucleosides of the modified crRNA comprise independently selected modified sugar moieities.

195. The compound of any of claim 91-107, 140-146, or 180-192, wherein the four 3′-terminal nucleosides of the modified crRNA comprise independently selected modified sugar moieities.

196. The compound of any of claim 91-107, 140-146, or 180-192, wherein the five 3′-terminal nucleosides of the modified crRNA comprise independently selected modified sugar moieities.

197. The compound of any of claim 77 or 193-196, wherein the modified sugar moieties of the 3′-terminal modified nucleosides are selected from among 2′-H(H), 2′-O-methyl, 2′-F, cEt, and LNA modified sugar moieties.

198. The compound of claim 197, wherein the modified sugar moieties of the 3′-terminal modified nucleosides are selected from among 2′-H(H), 2′-O-methyl, and cEt modified sugar moieties.

199. The compound of claim 197, wherein the modified sugar moieties of the 3′-terminal modified nucleosides are selected from among 2′-H(H) and 2′-O-methyl modified sugar moieties.

200. The compound of claim 197, wherein the modified sugar moieties of the 3′-terminal modified nucleosides are selected from among cEt and LNA modified sugar moieties.

201. The compound of any of claim 82-107, 140-146, or 180-200, wherein the 1st nucleoside from the 5′-end of the target recognition portion comprises a 2′-H(H) or 2′-F modified sugar moiety.

202. The compound of any of claim 82-107, 140-146, or 180-201, wherein the 8th nucleoside from the 5′-end of the target recognition portion comprises a 2′-H(H) or 2′-F modified sugar moiety.

203. The compound of any of claim 82-107, 140-146, or 180-202, wherein the 9th nucleoside from the 5′-end of the target recognition portion comprises a 2′-H(H) or 2′-F modified sugar moiety.

204. The compound of any of claim 91-107, 140-146, or 180-203, wherein the modified crRNA comprises at least three of the following features:
a. two linker nucleosides linked to the 5′-end of the CRISPR recognition portion of the modified crRNA;
b. 1^{st}, 8^{th}, and/or 9^{th} nucleoside from the 5′-end of the target recognition portion of the modified crRNA independently comprising 2′-F or 2′-H(H) modified sugar moiety;
c. at least one terminal phosphorothioate internucleoside linkage at each of the 3′ and 5′ termini of the modified crRNA
d. each nucleoside of the CRISPR recognition portion comprising an unmodified sugar moiety
e. one to five 3′-terminal nucleosides of the modified crRNA comprising independently selected modified sugar moieities

205. The compound of any of claim 1-107, 140-146, or 180-204, wherein the modified crRNA is a salt.

206. A pharmaceutical composition comprising the compound of any of claims 180-205.

207. The pharmaceutical composition of any of claim 108, 147, or 206, wherein the pharmaceutical composition comprises a ribonucleoprotein complex.

208. The pharmaceutical composition of claim 207, wherein the ribonucleoprotein complex comprises a Cpf1 nuclease and the compound comprising the modified crRNA.

209. A method comprising contacting a cell with the compound or composition of any of claims 180-208.

210. A method comprising contacting a cell with the compound or composition of any of claims 180-207, wherein the cell expresses a Cpf1 nuclease.

211. A method comprising contacting a cell with the compound or composition of any of claims 180-207 and a plasmid that encodes a Cpf1 nuclease.

212. A method comprising contacting a cell with the compound or composition of any of claims 180-207 and an mRNA that encodes a Cpf1 nuclease.

213. The method of any of claims 209-212, wherein the modified crRNA is taken up by the cell in the absence of a transfection reagent.

214. The method of any of claims 209-213, wherein the cell is in an animal.

215. A method comprising administering to an animal the compound or composition of any of claims 180-208.

216. The method of claim 215, wherein the administration is subcutaneous.

217. The method of claim 215, wherein the administration is intrathecal.

218. The method of any of claims 215-217 comprising administering a plasmid that encodes a Cpf1 nuclease.

219. The method of any of claims 215-217 wherein the animal expresses a Cpf1 nuclease.

220. The method of claim 211 or 218, wherein the plasmid is delivered to cells within the animal via an adeno-associated virus (AAV).

221. The method of claim 211 or 218, wherein the plasmid is delivered to cells within the animal via a lentivirus.

222. The method of any of claims 209-221, wherein a target gene is edited.

223. The method of claim 222, wherein the modified crRNA is degraded in a cell after the target gene is edited in the cell.

224. The method of any of claim 210-212 or 215-223, wherein the Cpf1 nuclease does not exhibit nuclease activity in the absence of the modified crRNA.

225. The method of any of claims 209-224 comprising contacting the cell with a second compound that degrades or inhibits the activity or expression of the modified crRNA or a Cpf1 nuclease.

226. The method of claim 225, wherein the cell is contacted with the second compound after a target gene has been edited.

227. The method of claim 225 or 226, wherein the second compound comprises an oligonucleotide that is complementary to the modified crRNA.

228. The method of claim 225 or 226, wherein the second compound comprises a crRNA that targets a Cpf1 nuclease gene.

229. The method of claim 225 or 226, wherein the second compound comprises an oligonucleotide that is complementary to a Cpf1 transcript.

230. The method of claim 228 or 229, wherein the expression of the Cpf1 nuclease is inhibited.

231. The method of any of claims 214-230, wherein the animal is a human.

232. The method of any of claims 209-231, wherein editing of at least one off-target gene is reduced relative to editing the at least one off-target gene when unmodified crRNA or a compound comprising more than 45 nucleosides is used in place of the modified crRNA.

233. The method of any of claim 215 or 218-232, wherein the administration is intravitreal.

234. The method of any of claims 209-213, wherein the cell is a plant cell.

235. The method of any of claims 209-214, wherein the cell is a T-cell.

236. A method of treating a disease in an individual comprising administering the compound of any of claims 180-205 or the composition of any of claims 206-208 to the individual.

237. A method of treating a disease in an individual comprising administering the compound of any of claims 180-205 or the composition any of claims 206-208 to the individual, thereby treating the disease in the individual.

238. Use of the compound of any of claims 180-205 or the composition of any of claims 206-208 for the treatment of a disease.

239. Use of the compound of any of claims 180-205 or the composition of any of claims 206-208 for preparation of a medicament.

240. A method of administering the compound of any of claims 180-205 or the composition of any of claims 206-208 to an animal, and harvesting an organ from the animal for transplantation into a human.

241. The compound of any of claim 1-107, 140-146, or 180-205, wherein the CRISPR recognition portion of the modified crRNA comprises at least one modified sugar moiety.

242. The compound of claim 241, wherein the at least one modified sugar moiety of the CRISPR recognition portion is a linearly modified sugar moiety.

243. The compound of claim 241, wherein the at least one modified sugar moiety of the CRISPR recognition portion is a bicyclic sugar moiety.

244. The compound of claim 243, wherein the bicyclic sugar moiety is cEt or LNA.

245. The compound of claim 243, wherein the bicyclic sugar moiety is cEt.

246. The compound of any of claims 241-245, wherein the 2nd nucleoside from the 3′-end of the CRISPR recognition portion comprises the at least one modified sugar moiety.

247. The compound of any of claims 241-245, wherein the 3rd nucleoside from the 3′-end of the CRISPR recognition portion comprises the at least one modified sugar moiety.

248. The compound of any of claims 241-245, wherein the 4th nucleoside from the 3′-end of the CRISPR recognition portion comprises the at least one modified sugar moiety.

249. The compound of any of claims 241-245, wherein the 5th nucleoside from the 3′-end of the CRISPR recognition portion comprises the at least one modified sugar moiety.

250. The compound of any of claims 241-245, wherein the 6th nucleoside from the 3′-end of the CRISPR recognition portion comprises the at least one modified sugar moiety.

251. The compound of any of claims 241-245, wherein the 7th nucleoside from the 3′-end of the CRISPR recognition portion comprises the at least one modified sugar moiety.

252. The compound of any of claims 241-245, wherein the 8th nucleoside from the 3′-end of the CRISPR recognition portion comprises the at least one modified sugar moiety.

253. The compound of any of claims 241-245, wherein the 9th nucleoside from the 3′-end of the CRISPR recognition portion comprises the at least one modified sugar moiety.

254. The compound of any of claims 241-245, wherein the 11th nucleoside from the 3′-end of the CRISPR recognition portion comprises the at least one modified sugar moiety.

255. The compound of any of claims 241-245, wherein the 12th nucleoside from the 3′-end of the CRISPR recognition portion comprises the at least one modified sugar moiety.

256. The compound of any of claims 241-245, wherein the 13th nucleoside from the 3′-end of the CRISPR recognition portion comprises the at least one modified sugar moiety.

257. The compound of any of claims 241-245, wherein the 18^{th} nucleoside from the 3′-end of the CRISPR recognition portion comprises the at least one modified sugar moiety.

258. The compound of any of claims 241-245, wherein the 11^{th} and 12^{th} nucleosides from the 3′-end of the CRISPR recognition portion each comprise a modified sugar moiety.

259. The compound of any of claims 241-258, wherein the 1^{st} nucleoside from the 3′-end of the CRISPR recognition portion comprises an unmodified sugar moiety.

260. The compound of any of claims 241-259, wherein the 10^{th} nucleoside from the 3′-end of the CRISPR recognition portion comprises an unmodified sugar moiety.

261. The compound of any of claims 241-260, wherein the 14^{th} nucleoside from the 3′-end of the CRISPR recognition portion comprises an unmodified sugar moiety.

262. The compound of any of claims 241-261, wherein the 15^{th} nucleoside from the 3′-end of the CRISPR recognition portion comprises an unmodified sugar moiety.

263. The compound of any of claims 241-262, wherein the 16^{th} nucleoside from the 3′-end of the CRISPR recognition portion comprises an unmodified sugar moiety.

264. The compound of any of claims 241-263, wherein the 17^{th} nucleoside from the 3′-end of the CRISPR recognition portion comprises an unmodified sugar moiety.

265. The compound of any of claims 241-264, wherein the 1st nucleoside from the 5′-end of the CRISPR recognition portion comprises an unmodified sugar moiety.

266. The compound of any of claims 241-265, wherein the 2nd nucleoside from the 5′-end of the CRISPR recognition portion comprises an unmodified sugar moiety.

267. The compound of any of claims 241-266, wherein the 3rd nucleoside from the 5′-end of the CRISPR recognition portion comprises an unmodified sugar moiety.

268. The compound of any of claim 1-107, 140-146, 180-205, or 241-267, wherein the 14^{th} nucleoside from the 5′-end of the target recognition portion comprises a modified sugar moiety.

269. The compound of any of claim 1-107, 140-146, 180-205, or 241-268, wherein the 15^{th} nucleoside from the 5′-end of the target recognition portion comprises a modified sugar moiety.

270. The compound of any of claim 1-107, 140-146, 180-205, or 241-269, wherein the 16^{th} nucleoside from the 5′-end of the target recognition portion comprises a modified sugar moiety.

271. The compound of any of claims 268-270, wherein each modified sugar moiety at position 14, 15, and/or 16 from the 5′-end of the target recognition portion is a linearly modified sugar moiety.

272. The compound of claim 271, wherein each modified sugar moiety at position 14, 15, and/or 16 from the 5′-end of the target recognition portion is independently selected from 2′-H(H) and 2′-F modified sugar moieties.

273. The compound of claim 272, wherein each modified sugar moiety at position 14, 15, and/or 16 from the 5′-end of the target recognition portion is a 2′-H(H) modified sugar moiety.

274. The compound of any of claim 1-107, 140-146, 180-205, or 241-273, wherein the modified crRNA comprises at least three of the following features:
(a) two linker nucleosides linked to the 5′-end of the CRISPR recognition portion of the modified crRNA;
(b) 1^{st}, 8^{th}, and/or 9^{th} nucleoside from the 5′-end of the target recognition portion of the modified crRNA independently comprising 2′-F or 2′-H(H) modified sugar moiety;
(c) at least one terminal phosphorothioate internucleoside linkage at each of the 3′ and 5′ termini of the modified crRNA
(d) at least one nucleoside at position 5, 6, 7, 8, 11, or 12 from the 3′-end of the CRISPR recognition portion comprises a modified sugar moiety
(e) one to five 3′-terminal nucleosides of the modified crRNA comprising independently selected modified sugar moieities

275. The compound of claim 274, wherein the modified crRNA comprises features (a), (c), and (e).

276. The compound of claim 274, wherein the modified crRNA comprises features (a), (c), and (d).

277. The compound of claim 274, wherein the modified crRNA comprises features (a), (b), and (c).

278. The compound of claim 274, wherein the modified crRNA comprises features (a), (b), (c), and (e).

279. The compound of claim 274, wherein the modified crRNA comprises features (a), (c), (d), and (e).

280. The compound of claim 274, wherein the modified crRNA comprises features (a), (b), (c), (d), and (e).

281. A pharmaceutical composition comprising the compound of any of claims 241-280.

282. The pharmaceutical composition of claim 281, wherein the pharmaceutical composition comprises a ribonucleoprotein complex.

283. The pharmaceutical composition of claim 282, wherein the ribonucleoprotein complex comprises a Cpf1 nuclease and the compound comprising the modified crRNA.

284. A method comprising contacting a cell with the compound or composition of any of claims 241-283.

285. A method comprising contacting a cell with the compound or composition of any of claims 241-283, wherein the cell expresses a Cpf1 nuclease.

286. A method comprising contacting a cell with the compound or composition of any of claims 241-283 and a plasmid that encodes a Cpf1 nuclease.

287. A method comprising contacting a cell with the compound or composition of any of claims 241-283 and an mRNA that encodes a Cpf1 nuclease.

288. The method of any of claims 284-287, wherein the modified crRNA is taken up by the cell in the absence of a transfection reagent.

289. The method of any of claims 284-288, wherein the cell is in an animal.

290. A method comprising administering to an animal the compound or composition of any of claims 241-283.

291. The method of claim 290, wherein the administration is subcutaneous.

292. The method of claim 290, wherein the administration is intrathecal.

293. The method of any of claims 290-292 comprising administering a plasmid that encodes a Cpf1 nuclease.

294. The method of any of claims 290-292 wherein the animal expresses a Cpf1 nuclease.

295. The method of claim 286 or 293, wherein the plasmid is delivered to cells within the animal via an adeno-associated virus (AAV).

296. The method of claim 286 or 293, wherein the plasmid is delivered to cells within the animal via a lentivirus.

297. The method of any of claims 284-296, wherein a target gene is edited.

298. The method of claim 297, wherein the modified crRNA is degraded in a cell after the target gene is edited in the cell.

299. The method of any of claim 285-287 or 293-298, wherein the Cpf1 nuclease does not exhibit nuclease activity in the absence of the modified crRNA.

300. The method of any of claims 284-299 comprising contacting the cell with a second compound that degrades or inhibits the activity or expression of the modified crRNA or a Cpf1 nuclease.

301. The method of claim 300, wherein the cell is contacted with the second compound after a target gene has been edited.

302. The method of claim 300 or 301, wherein the second compound comprises an oligonucleotide that is complementary to the modified crRNA.

303. The method of claim 300 or 301, wherein the second compound comprises a crRNA that targets a Cpf1 nuclease gene.

304. The method of claim 300 or 301, wherein the second compound comprises an oligonucleotide that is complementary to a Cpf1 transcript.

305. The method of claim 303 or 304, wherein the expression of the Cpf1 nuclease is inhibited.

306. The method of any of claims 289-305, wherein the animal is a human.

307. The method of any of claims 284-306, wherein editing of at least one off-target gene is reduced relative to editing the at least one off-target gene when unmodified crRNA or a compound comprising more than 45 nucleosides is used in place of the modified crRNA.

308. The method of any of claim 290 or 293-297, wherein the administration is intravitreal.

309. The method of any of claims 284-288, wherein the cell is a plant cell.

310. The method of any of claims 284-289, wherein the cell is a T-cell.

311. A method of treating a disease in an individual comprising administering the compound of any of claims 241-280 or the composition of any of claims 281-283 to the individual.

312. A method of treating a disease in an individual comprising administering the compound of any of claims 241-280 or the composition any of claims 281-283 to the individual, thereby treating the disease in the individual.

313. Use of the compound of any of claims 241-280 or the composition of any of claims 281-283 for the treatment of a disease.

314. Use of the compound of any of claims 241-280 or the composition of any of claims 281-283 for preparation of a medicament.

315. A method of administering the compound of any of claims 241-280 or the composition of any of claims 281-283 to an animal, and harvesting an organ from the animal for transplantation into a human.

316. The pharmaceutical composition of any of claim 108, 147, 206, or 281 comprising a liposome or lipid nanoparticle.

317. The pharmaceutical composition of any of claim 108, 147, 206, 281, or 316 comprising mRNA that encodes a Cpf1 nuclease.

318. The pharmaceutical composition of claim 317, wherein the compound comprising the modified crRNA and the mRNA encoding a Cpf1 nuclease are contained with a liposome or lipid nanoparticle.

319. The method of any of claim 212-214, 151-153, 212-214, or 287-289, wherein the mRNA encoding the Cpf1 nuclease and the compound comprising the modified crRNA are contained within a liposome or lipid nanoparticle.

320. A method of treating a disease in an individual comprising administering the pharmaceutical composition of any of claims 316-318 to the individual.

321. A method of treating a disease in an individual comprising administering the pharmaceutical composition of any of claims 316-318 to the individual, thereby treating the disease in the individual.
